SEQUENCE LISTING (29 sequences)

SEQ ID NO: 1 - DNA, 21 bp, Artificial Sequence
  302F-universal-Tm54
cacactggractgagayacgg

SEQ ID NO: 2 - DNA, 19 bp, Artificial Sequence
  333F-universal-Tm55-2X
actcctacgggaggcwgca

SEQ ID NO: 3 - DNA, 18 bp, Artificial Sequence
  528F-universal-Tm57-4X
gtgccagcagyygcggta

SEQ ID NO: 4 - DNA, 21 bp, Artificial Sequence
  802R-universal-Tm52-8X
gaytacyrgggtatctaatcc

SEQ ID NO: 5 - DNA, 22 bp, Artificial Sequence
  897R-universal-Tm53-6X
ccccgtcaatthmtttgagttt

SEQ ID NO: 6 - DNA, 22 bp, Artificial Sequence
  1072R-universal-Tm55-2X
cgttrcgggacttaacccaaca

SEQ ID NO: 7 - DNA, 18 bp, Artificial Sequence
  1166R-Tm53-4X
tcrtccycaccttcctcc

SEQ ID NO: 8 - DNA, 19 bp, Artificial Sequence
  1380R-Tm52-8X
yccgrgaacgtattcacsg

SEQ ID NO: 9 - DNA, 17 bp, Artificial Sequence
  18S-304F-2-fold-Tm50.7
ggtattggcctaccgrg

SEQ ID NO: 10 - DNA, 19 bp, Artificial Sequence
  18S-413F-0-fold-Tm51.1
tacccaatcctgacacagg

SEQ ID NO: 11 - DNA, 21 bp, Artificial Sequence
  18S-877R-2-fold-Tm53.4
gctttcgcagtrgttcgtctt

SEQ ID NO: 12 - DNA, 20 bp, Artificial Sequence
  18S-931R-0-fold-Tm51.8
cgtcttcgatcccctaactt

SEQ ID NO: 13 - DNA, 19 bp, Artificial Sequence
  18S-1018F-0-fold-Tm51.1
gactccttcagcaccttga

SEQ ID NO: 14 - DNA, 21 bp, Artificial Sequence
  18S-1619R-0-fold-Tm50.5
cgaataattcaccggatcact

SEQ ID NO: 15 - DNA, 26 bp, Artificial Sequence
  18S-1679R-0-fold-Tm51.7
agttttgtgaaccttatcacttaaag

SEQ ID NO: 16 - DNA, 17 bp, Artificial Sequence
  Mycobacterium-rpoB-259F-4XTm47.1-51.9
cacggcaayaagggygt

SEQ ID NO: 17 - DNA, 18 bp, Artificial Sequence
  Mycobacterium-rpoB-274F-6XTm45.8-50.3
gtbatcggcaagatyctc

SEQ ID NO: 18 - DNA, 17 bp, Artificial Sequence
  Mycobacterium-rpoB-697R-1XTm51.9
tcaccgggtacgggaac

SEQ ID NO: 19 - DNA, 17 bp, Artificial Sequence
  Mycobacterium-rpoB-755R-4XTm47.1-49.5
gcgtgratcttgtcrtc

SEQ ID NO: 20 - DNA, 18 bp, Artificial Sequence
  Mycobacterium-hsp65-322F-2XTm=52.6
tacgagaagatcggcgcy

SEQ ID NO: 21 - DNA, 18 bp, Artificial Sequence
  Mycobacterium-hsp65-282F-1XTm=52.6
ggtgtgtccatcgccaag

SEQ ID NO: 22 - DNA, 18 bp, Artificial Sequence
  Mycobacterium-hsp65-650R-3XTm=51.9
ctcgttgccvaccttgtc

SEQ ID NO: 23 - DNA, 18 bp, Artificial Sequence
  Mycobacterium-hsp65-670R-2XTm=52.6
ctcgacggtgatgacrcc

SEQ ID NO: 24 - DNA, 16 bp, Artificial Sequence
  Fungal-18S-SSU-forward1-4X
gtacacackccygtcg

SEQ ID NO: 25 - DNA, 23 bp, Artificial Sequence
  Fungal-18S-SSU-forward2-6X
tgyaattdttgctcttcaacgag

SEQ ID NO: 26 - DNA, 20 bp, Artificial Sequence
  Fungal-296R-4X
gctscgttcttcatcgatsc

SEQ ID NO: 27 - DNA, 21 bp, Artificial Sequence
  Fungal-350R-4X
gttcaagaytcratgattcac

SEQ ID NO: 28 - DNA, 20 bp, Artificial Sequence
  Fungal-296R-Pneumocystis
gccacgttcttcatcgacgc

SEQ ID NO: 29 - DNA, 22 bp, Artificial Sequence
  Fungal-350R-Pneumocystis
gttcaaaaattcgatgattcac

(19) United States
(12) Patent Application Publication
(10) Pub. No.: US 2023/0167510 A1
(43) Pub. Date: Jun. 1, 2023

(54) UNIVERSAL PRIMERS FOR DETECTION OF BACTERIA, FUNGI AND EUKARYOTIC MICROORGANISMS

(71) Applicant: The Regents of the University of California, Oakland, CA (US)

(72) Inventor: Charles Chiu, Oakland, CA (US)

(21) Appl. No.: 17/920,379

(22) PCT Filed: Apr. 22, 2021
(86) PCT No.: PCT/US2021/028629

§ 371 Date: Oct. 20, 2022

Related U.S. Application Data
(60) Provisional application No. 63/013,892, filed on Apr. 22, 2020.

(51) Int. Cl. C12Q 1/689; C12Q 1/6853; C12Q 1/6895; C07H 21/00

(52) U.S. Cl. CPC C12Q 1/689; C12Q 1/6853; C12Q 1/6895; C07H 21/00; C12Q 2600/16

(57) ABSTRACT

Methods, compositions and kits for detection of a taxon of microorganisms in a sample are provided.

### CROSS REFERENCE TO RELATED APPLICATIONS

This application claims priority under 35 U.S.C. § 119 from Provisional Application Ser. No. 63/013,892, filed Apr. 22, 2020, the disclosures of which are incorporated herein by reference for all purposes.

### GOVERNMENT LICENSE RIGHTS

This invention was made with Government support under Grant No: W81XWH-17-1-0681, awarded by the Department of Defense and Grant No. R33AI120977, awarded by the National Institutes of Health. The Government has certain rights in the invention.

### INCORPORATION BY REFERENCE OF SEQUENCE LISTING

Accompanying this filing is a Sequence Listing entitled “Sequence-Listing ST25.txt”, created on Apr. 22, 2021 and having 6,079 bytes of data, machine formatted on IBM-PC, MS-Windows operating system. The sequence listing is hereby incorporated herein by reference in its entirety for all purposes.

### FIELD

The disclosure relates to the field of genomics and diagnostics, and more particularly to the detection and genomic characterization of microorganisms in a sample.

### BACKGROUND

Sequencing of conserved regions of ribosomal RNA, including the 16S (SSU, small subunit), 23S (LSU, large subunit) of bacteria, ITS regions of fungi, and the corresponding 18S (SSU) and 28S (LSU) regions of fungi and parasites, is critical to several applications, including clinical diagnostic metagenomics, microbiome analysis, forensics, and public health screening (summarized in Chiu and Miller, Nature Review Genetics, 20:341-355, 2019). For decades, standardized published primer sets have been used for this purpose (see Janda et al., J. Clin. Microbiol., 45(9):2761-2764, 2007; Salipant et al., PloS One, 8(5):e65226, 2013). However, these “historical” primer sets have been based on limited databases and targets. The sensitivity of these primer sets in general for clinical microbial diagnostics has been called into question (Payne et al., Canadian J. of Infect. Dis. And Med. Microbiol., vol. 2016:1-7, 2016)—for 2 main reasons: (1) contaminating bacterial DNA that can decrease sensitivity for target DNA, and (2) concerns regarding the “universality” of the primers used. There are now many documented instances where clinical 16S sequencing failed to diagnose the cause of a bacterial infection, particularly if it is rare or unusual (Wilson et al., NEJM, 370:2408-2417, 2014).

### SUMMARY

The disclosure provides an isolated oligonucleotide selected from the sequences consisting of: (i) a sequence comprising any one of SEQ ID NO:1-29, having 1-5 nucleotides added or removed from the 5′ and/or 3′ ends; and (ii) a sequence consisting of any one of SEQ ID NO:1-29.

The disclosure also provides a composition for microbial detection, the composition comprising at least one oligonucleotide set forth in any one of SEQ ID NOs: 1-29. In one embodiment, the at least one oligonucleotide comprises at least two or more oligonucleotides. In another embodiment, the at least one oligonucleotide is selected from oligonucleotides having the sequence of SEQ ID NOs:1-7, 8, or any two or more of SEQ ID NOs:1-8. In still another or further embodiment, the composition comprising the sequence of SEQ ID NOs:1-7, 8, or any two or more of SEQ ID NOs:1-8 is used to detect bacteria. In yet another or further embodiment, the at least one oligonucleotide is selected from oligonucleotides having the sequence of SEQ ID NOs:9-14, 15, or any two or more of SEQ ID NOs:9-15. In still another or further embodiment, the composition comprising the sequence of SEQ ID NOs:9-14, 15, or any two or more of SEQ ID NOs:9-15 is used to detect babesia. In yet another or further embodiment, the at least one oligonucleotide is selected from oligonucleotides having the sequence of SEQ ID NOs:16-22, 23, or any two or more of SEQ ID NOs:16-23. In still another or further embodiment, the composition comprising the sequence of SEQ ID NOs:16-22, 23, or any two or more of SEQ ID NOs:16-23 is used to detect mycobacteria. In yet another or further embodiment, the at least one oligonucleotide is selected from oligonucleotides having the sequence of SEQ ID NOs:24-28, 29, or any two or more of SEQ ID NOs:24-29. In still another or further embodiment, the composition comprising the sequence of SEQ ID NOs:24-28, 29, or any two or more of SEQ ID NOs:24-29 is used to detect fungi.

The disclosure provides a composition for detecting a microbe selected from the group consisting of bacteria, mycobacteria, babesia, fungi and any combination thereof, the composition comprising at least one primer having a sequence selected from the group consisting of SEQ ID NO:1-29 and any combination thereof.

The disclosure also provides a method of detecting the presence of a bacterial species in a sample, the method comprising contacting the sample with at least one universal primer having a sequence set forth in any one of SEQ ID NOs: 1-8.

The disclosure provides a method of detecting the presence of a babesia species in a sample, the method comprising contacting the sample with at least one universal primer having a sequence set forth in any one of SEQ ID NOs: 9-15.

The disclosure also provides a method of detecting the presence of a mycobacterial species in a sample, the method comprising contacting the sample with at least one universal primer having a sequence set forth in any one of SEQ ID NOs: 16-23.

The disclosure provides a method of detecting the presence of a fungal species in a sample, the method comprising contacting the sample with at least one universal primer having a sequence set forth in any one of SEQ ID NOs: 24-29.

The disclosure also provides a method for determining microbial content in a sample, said method comprising amplifying a target nucleotide sequence which is substantially conserved amongst two or more species of microorganisms, said amplification being for a time and under conditions sufficient to generate a level of an amplification product such that the presence of the microbe can be detected, wherein the method uses at least one primer selected from SEQ ID NOs:1-29. In one embodiment, the target nucleotide sequence is DNA. In yet another embodiment, the target nucleotide sequence is RNA. In yet another or further embodiment, the target nucleotide sequence is ribosomal DNA (rDNA). In yet another embodiment, the target nucleotide sequence is ribosomal RNA (rRNA). In still another or further embodiment, the rDNA is 16S rDNA. In yet another or further embodiment, the rRNA is 16S rRNA. In another embodiment, the sample is a biological, medical, agricultural, industrial or environmental sample. In another embodiment, the amplification is by polymerase chain reaction (PCR). In still another embodiment, the amplification primer comprises a primer having the sequence selected from SEQ ID NO:1-8 or a sequence having from 1-5 additional nucleotides at the 5′ and/or 3′ end of any of the sequence of SEQ ID NO:1-8 and wherein the microbial content is bacteria. In yet another embodiment, the amplification primer comprises a primer having the sequence selected from SEQ ID NO:9-15 or a sequence having from 1-5 additional nucleotides at the 5′ and/or 3′ end of any of the sequence of SEQ ID NO:9-15 and wherein the microbial content is babesia. In another embodiment, the amplification primer comprises a primer having the sequence selected from SEQ ID NO:16-23 or a sequence having from 1-5 additional nucleotides at the 5′ and/or 3′ end of any of the sequence of SEQ ID NO:16-23 and wherein the microbial content is mycobacteria. In another embodiment, the amplification primer comprises a primer having the sequence selected from SEQ ID NO:24-29 or a sequence having from 1-5 additional nucleotides at the 5′ and/or 3′ end of any of the sequence of SEQ ID NO:24-29 and wherein the microbial content is fungi.

The disclosure also provides a kit in compartmental form, said kit comprising a compartment adapted to contain one or more primers having a sequence selected from SEQ ID NOs:1-29, and any combination thereof, capable of participating in an amplification reaction of DNA comprising or associated with 16S rDNA or 16S rRNA, and optionally another compartment adapted to contain reagents to conduct an amplification reaction.

These and other embodiments are described in more detail below.

### DETAILED DESCRIPTION

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Spring Harbor Lab Press (Cold Spring Harbor, N.Y. 1989), both of which are incorporated herein by reference. All patents, patent applications, and publications mentioned herein are incorporated herein by reference in their entireties for all purposes.

The term “a”, “an” or “the” is intended to mean “one or more”, e.g., a pathogen refers to one or more pathogenic microorganisms unless otherwise made clear from the context of the text.

The term “comprise,” and variations thereof such as “comprises” and “comprising,” when preceding the recitation of a step or an element, are intended to mean that the addition of further steps or elements is optional and not excluded.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Any methods and reagents similar or equivalent to those described herein can be used in the practice of the disclosed methods and compositions.

As used herein, the term “amplifying” refers to the process of synthesizing nucleic acid molecules that are complementary to one (or both strands) of a template nucleic acid molecule. Amplifying a nucleic acid molecule typically includes denaturing the template nucleic acid, particularly if the template nucleic acid is double-stranded, annealing one or more primers to the template nucleic acid at a temperature that is below the melting temperatures of the primers, and enzymatically elongating from the primers to generate an amplification product. Generally, synthesis initiates at the 3′ end of a primer and proceeds in a 5′ to 3′ direction along the template nucleic acid strand. Amplification typically requires the presence of deoxyribonucleoside triphosphates, a polymerase enzyme (e.g., DNA or RNA polymerase or T7 for in vitro transcription in TMA) and an appropriate buffer and/or co-factors for optimal activity of the polymerase enzyme (e.g., MgCl₂ and/or KCl).

As used herein, the term “complement thereof” or “complementary” refers to a nucleic acid molecule that is optionally the same length as a target molecule of interest and possesses a structural (e.g., nucleotide) composition that is complementary (i.e., capable of conventional hydrogen base pairing) with the target molecule of interest, unless otherwise specified. Substantial complementarity refers to a nucleic acid molecule that is optionally the same length as the target molecule of interest but is greater than 90% complementary and less than 100% complementary to the target molecule of interest.

With respect to the term “different taxon of pathogens”, the term is distinct from the “particular taxon of pathogens”. Here, the different taxon of pathogenic microorganisms does not overlap with the particular taxon of pathogens. For example, if a particular taxon of pathogenic microorganisms includes the family of Flavivirus, the different taxon of pathogenic microorganisms does not include Flavivirus but can include another family of viruses, such as Alphaviruses, bacterial, fungal, archaea, algal, protozoan, and/or parasitic pathogens. If the particular taxon of pathogenic microorganisms and different taxon of pathogenic microorganisms are from the same domain (e.g., bacterial domain), the two taxa identified by the method are distinct.

As used herein, the terms “extension”, “extend” or “elongation” when used with respect to nucleic acid molecules refers to a biological process by which additional nucleotides (or nucleotide analogs) are incorporated into nucleic acid molecules. For example, a nucleic acid can be extended by a nucleotide incorporating enzyme, such as a polymerase or reverse transcriptase that typically adds sequentially, a nucleotide to the 3′ terminal end of the nucleic acid molecule (e.g., the freely available 3′ —OH group).

As used herein, “hybridization”, “hybridizing”, “anneal” and “annealing”, and the like, refer to a process of combining two complementary (or substantially complementary (e.g., at least 90%) single-stranded DNA or RNA molecules so as to form a double-stranded molecule (DNA/DNA, DNA/RNA, RNA/RNA) through conventional hydrogen base pairing. Hybridization stringency is typically determined by the hybridization temperature and salt concentration of the hybridization buffer; e.g., high temperature and low salt provide high stringency hybridization conditions. Examples of salt concentration ranges and temperature ranges for different hybridization conditions are as follows: high stringency, approximately 0.01 M to approximately 0.05 M salt, hybridization temperature 5° C. to 10° C. below Tₘ; moderate stringency, approximately 0.16 M to approximately 0.33 M salt, hybridization temperature 20° C. to 29° C. below Tₘ; and low stringency, approximately 0.33 M to approximately 0.82 M salt, hybridization temperature 40° C. to 48° C. below Tₘ of duplex nucleic acids is calculated by standard methods well-known in the art (see, e.g., Maniatis, T., et al., Molecular Cloning: A Laboratory Manual, Cold Spring Harbor Laboratory Press: New York (1982); Casey, J., et al., Nucleic Acids Research 4:1539-1552 (1977); Bodkin, D. K., et al., Journal of Virological Methods 10(1):45-52 (1985); Wallace, R. B., et al., Nucleic Acids Research 9(4):879-894 (1981)). Algorithm prediction tools to estimate Tₘ are also publicly available (see, e.g., [http://] [tmcalculator.neb.com]). High stringency conditions for hybridization typically refer to conditions under which a nucleic acid molecule having complementarity (or substantial complementarity, e.g., greater than 90%, 95%, 98%, 99% complementarity) to a target sequence predominantly hybridizes with the target sequence and does not hybridize to non-target or off-target sequences.

In some embodiments, hybridizing refers to the annealing of a primer to a complementary (or substantially complementary (e.g., greater than 90% complementary)) template (or target) RNA or DNA sequence obtained from a pathogen. In another embodiment, hybridizing can include annealing at least one probe to an amplification product (e.g., cDNA molecule) derived from a pathogen. Hybridization conditions typically include a temperature below the melting temperature of the primers or probes to reduced non-specific hybridization of the primers/probes. Accordingly, in some embodiments of the disclosure, hybridization conditions are of moderate stringency or high stringency.

As used herein, the terms “identical” or “percent identity” in the context of two or more nucleic acid sequences, refers to two or more sequences that are the same or have a specified percentage of nucleotides that are the same (i.e., identical), when compared and aligned for maximum correspondence, e.g., as measured using one of the sequence comparison algorithms or by visual inspection. An exemplary algorithm that is suitable for determining percent sequence identity and sequence similarity is the BLAST program, which are described in Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:113-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656.

Other exemplary multiple sequence alignment computer programs include MAFFT [https://] [mafft.cbrc.jp/alignment/software/]), MUSCLE [https://] [www.ebi.ac.uk/Tools/msa/muscle/]), and CLUSTALW [https://] [www.ebi.ac.uk/Tools/msa/clustalw2/]). Percent identity between two nucleic acid sequences is generally calculated using standard default parameters of the various methods or computer programs. A high degree of sequence identity, as used herein, between two nucleic acid molecules is typically at least 90% identity, at least 91% identity, at least 92% identity, at least 93% identity, at least 94% identity, at least 95% identity, at least 96% identity, at least 97% identity, at least 98% identity, at least 99% identity, at least 99.5% identity, or any range of percent identity that includes or is between any two of the foregoing percentages (e.g., between 90% identity and 100% identity, between 95% identity and 98% identity, etc.). A moderate degree of sequence identity, as used herein, between two nucleic acid molecules is typically at least 80% identity, at least 82% identity, at least 83% identity, at least 84% identity, at least 85% identity, at least 86% identity, at least 87% identity, at least 88% identity, at least 89% identity, or any range of percent identity that includes or is between any two of the foregoing percentages (e.g., between 80% identity and 90% identity, between 85% identity and 89% identity, etc.). A low degree of sequence identity, as used herein, between two nucleic acid molecules is typically at least 50% identity, at least 55% identity, at least 60% identity, at least 65% identity, at least 70% identity, at least 75% identity, at least 79% identity, or any range of percent identity that includes or is between any two of the foregoing percentages (e.g., between 50% identity and 70% identity, 55% identity and 75% identity). For example, a sample from a subject, (e.g., suspected of being infected with Zika virus) can have a high degree of sequence identity to a reference taxon of pathogenic microorganisms (e.g., Flavivirus) and a low degree of sequence identity to bacterial pathogenic microorganisms (e.g., Streptococcus, Clostridium, Salmonella and Mycobacterium).

The term “microorganism” or “microbial organism” is used in its broadest sense and includes Gram negative aerobic bacteria, Gram positive aerobic bacteria, Gram negative microaerophillic bacteria, Gram positive microaerophillic bacteria, Gram negative facultative anaerobic bacteria, Gram positive facultative anaerobic bacteria, Gram negative anaerobic bacteria, Gram positive anaerobic bacteria, Gram positive asporogenic bacteria, Actinomycetes, fungal microorganism, protazoan microorganism and the like.

As used herein, a “modified nucleotide” or “nucleotide analog” in the context of an oligonucleotide, primer or probe, refers to incorporation of a non-naturally occurring nucleotide (e.g., a nucleotide other than A, G, T, C or U) within the oligonucleotide, primer or probe, and whereby incorporation of the modified nucleotide or nucleotide analog does not hinder or prevent nucleic acid extension or elongation under suitable amplification conditions. Examples of nucleic acid modifications are described in, e.g., U.S. Pat. No. 6,001,611. Other modified nucleotide substitutions may alter the stability of the oligonucleotide (e.g., modulate its Tₘ), or provide other desirable features (e.g., nuclease resistance).

As used herein, the terms “nucleic acid”, “polynucleotide” and “oligonucleotide” refer to a polymeric form of nucleotides. The nucleotides may be deoxyribonucleotides (DNA), ribonucleotides (RNA), analogs thereof, or combinations thereof, and may be of any length. Polynucleotides may perform any function and may have any secondary and tertiary structures (e.g., hairpins, stem loop structures). Oligonucleotides refer to polymeric form of nucleotides typically having much shorter lengths than polynucleotides (e.g., ≤50 nt). The terms encompass known analogs of natural nucleotides and nucleotides that are modified in the base, sugar and/or phosphate moieties. Preferably, analogs of a particular nucleotide have the same base-pairing specificity (e.g., an analog of A base pairs with T). An oligonucleotide may comprise one modified nucleotide or multiple modified nucleotides. Examples of modified nucleotides include fluorinated nucleotides, methylated nucleotides, and nucleotide analogs. The nucleotide structure may be modified before or after a polymer is assembled. The terms also encompass nucleic acids comprising modified backbone residues or linkages that are synthetic, naturally occurring, and non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA) and morpholino structures.

As used herein, the term “pathogen” refers to a virus, bacterium, protozoa, prion, archaea, fungus, algae, parasite, or other microbe (helminth) that causes or induces disease or illness in a subject or that may be found in biological and/or environmental samples. The term includes both the disease-causing organism per se and toxins produced by the pathogen (e.g., Shiga toxins) present in a sample. Detection of a pathogen as set forth in the methods disclosed herein includes detection of a portion of the genome of the pathogen or a nucleic acid molecule that is complementary or substantially complementary (i.e., at least 90% complementary) to a portion of the genome of the pathogen.

With respect to the term “particular taxon of pathogens”, the term refers to classification or taxonomy of pathogens. Accordingly, a “particular taxon of pathogens” can include pathogenic microorganisms classified at various levels of taxonomic rank, e.g., by Realm (Riboviria), Domain/SubRealm (e.g., Bacteria, Arachaea), by Kingdom (e.g., Protista, Fungi, etc.), by Phylum (e.g., Vira, Chlamydiae, etc.), by Class (e.g., Chlamydiales, Parachlamydiales, etc.), by Order (e.g., caudovirales, herpesvirales, ligamenvirales, mononegavirales, etc.), by Family (e.g., Reoviridae, Caliciviridae, Flaviviridae, Orthomyxoviridae, Picornaviridae, Togaviridae, Paramyxoviridae, Bunyaviridae, Rhabdoviridae, Filoviridae, Coronaviridae, Astroviridae, Bornaviridae, Arteriviridae, Hepeviridae, Retroviridae, etc.), or by Genus (e.g., Hepacivirus, flavivirus, pegivirus, pestivirus, etc.). Thus, “a particular taxon of pathogens” refers to a group of related species that share significant properties, but may differ in host range and virulence.

Bacteria and fungi are routinely classified or ranked based on different taxa corresponding to genus, family, and species identification. For example, fungal taxon contemplated by the disclosure include any of the fungal taxon provided in List 1 or List 2. It will be apparent to one of ordinary skill in the art that List 1 and List 2 are not exhaustive and are provided as exemplary lists.

List 1: Fungal Genera:

Anaeromyces, Caecomyces, Allomyces, Entyloma, Diskagma, Blastocladia, Funneliformis, Entylomella, Coelomomyces, Glomus (fungus), Fusidiurn, Heptameria, Holmiella, Homostegia, Hyalocrea, Hyalosphaera, Hypholoma, Hypobryon, Hysteropsis, Koordersiella, Karschia, Kirschsteiniothelia, Lembosiopeltis, Kullhemia, Kusanobotrys, Leptodothiorella, Lanatosphaera, Lasiodiplodia, Leveillina, Lepidopterella, Lepidostroma, Leptosphaerulina, Leptospora, Macrovalsaria, Lichenostigma, Licopolia, Massariola, Lopholeptosphaeria, Maireella, Microdothella, Macroventuria, Microcyclella, Mycoglaena, Melanodothis, Montagnella, Mycoporopsis, Moniliella, Mycopepon, Myriangiurn, Mycomicrothelia, Mycothyridium, Mytilostoma, Mycosphaerella, Mytilinidion, Neofusicoccum, Myriostigmella, Neocallimastix, Oomyces, Neopeckia, Orpinomyces, Ostreichnion, Ophiosphaerella, Paropodia, Passeriniella, Passerinula, Pedumispora, Peyronellaea, Phaeoacremonium, Phaeocyrtidula, Phaeoglaena, Phaeopeltosphaeria, Phaeoramularia, Phaeosperma, Phaneromyces, Phialophora, Philonectria, Phragmocapnias, Phragmosperma, Piedraia, Piromyces, Placocrea, Placostromella, Plagiostromella, Plejobolus, Pleostigma, Polychaeton, Pseudocercospora, Pseudocryptosporella, Pseudogymnoascus, Pseudothis, Pycnocarpon, Rhytidhysteron, Rhizophagus (fungus), Rhopographus, Rosellinula, Rhytisma, Robillardiella, Roussoellopsis, Rosenscheldia, Rostafinskia, Sarcopodium, Savulescua, Saksenaeaceae, Scolecobonaria, Scolicotrichum, Schizoparme, Semifissispora, Septoria, Scorias, Sphaceloma, Sphaerellothecium, Spathularia, Stagonosporopsis, Stenella (fungus), Sphaerulina, Stigmina (fungus), Stioclettia, Stigmidium, Sydowia, Tephromela, Stuartella, Teichosporella, Thalloloma, Taeniolella, Thalassoascus, Togninia, Teratosphaeria, Thyrospora, Thyridaria, Yarrowia, Wettsteinina, Valsaria, Ustilaginoidea, Yoshinagella, Wernerella (fungus), and Vismya.

List 2: Fungi Species:

Absidia corymbifera, Absidia ramose, Achorion gallinae, Actinomadura spp., Ajellomyces dermatididis, Aleurisma brasiliensis, Allersheria boydii, Arthroderma spp., Aspergillus flavus, Aspergillus fumigatu, Basidiobolus spp, Blastomyces spp, Cadophora spp, Candida albicans, Cercospora apii, Chrysosporium spp, Cladosporium spp, Cladothrix asteroids, Coccidioides immitis, Cryptococcus albidus, Cryptococcus gattii, Cryptococcus laurentii, Cryptococcus neoformans, Cunninghamella elegans, Dematium wernecke, Discomyces israelii, Emmonsia spp, Emmonsiella capsulate, Endomyces geotrichum, Entomophthora coronate, Epidermophyton floccosum, Filobasidiella neoformans, Fonsecaea spp., Geotrichum candidum, Glenospora khartoumensis, Gymnoascus gypseus, Haplosporangium parvum, Histoplasma, Histoplasma capsulatum, Hormiscium dermatididis, Hormodendrum spp., Keratinomyces spp, Langeronia soudanense, Leptosphaeria senegalensis, Lichtheimia corymbifera, Lobmyces loboi, Loboa loboi, Lobomycosis, Madurella spp., Malassezia furfur, Micrococcus pelletieri, Microsporum spp, Monilia spp., Mucor spp., Mycobacterium tuberculosis, Nannizzia spp., Neotestudina rosatii, Nocardia spp., Oidium albicans, Oospora lactis, Paracoccidioides brasiliensis, Petriellidium boydii, Phialophora spp., Piedraia hortae, Pityrosporum furfur, Pneumocystis jirovecii (or Pneumocystis carinii), Pullularia gougerotii, Pyrenochaeta romeroi, Rhinosporidium seeberi, Sabouraudites (Microsporum), Sartorya fumigate, Sepedonium, Sporotrichum spp., Stachybotrys, Stachybotrys chartarum, Streptomyce spp., Tinea spp., Torula spp, Trichophyton spp, Trichosporon spp, and Zopfia rosatii.

Additionally, bacterial taxon contemplated by the disclosure include any of the bacterial taxon provided in List 3 or List 4. It will be apparent to one of ordinary skill in the art that List 3 and List 4 are not exhaustive and are provided as exemplary lists.

List 3: Bacterial Genera: Heliobacter, Aerobacter, Rhizobium, Agrobacterium, Bacillus, Clostridium, Pseudomonas, Xanthomonas, Nitrobacteriaceae, Nitrobacter, Nitrosomonas, Thiobacillus, Spirillum, Vibrio, Bacteroides, Corynebacterium, Listeria, Escherichia, Klebsiella, Salmonella, Serratia, Shigella, Erwinia, Rickettsia, Chlamydia, Mycoplasma, Actinomyces, Streptomyces, Mycobacterium, Polyangium, Micrococcus, Staphylococcus, Lactobacillus, Diplococcus, Streptococcus, and Campylobacter.

List 4: Bacterial Species:

Actinomyces israelii, Bacillus anthracis, Bacillus cereus, Bartonella henselae, Bartonella quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsia, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Ureaplasma urealyticum, Vibrio cholerae, Yersinia pestis, Yersinia enterocolitica, and Yersinia pseudotuberculosis.

As used herein, the term “primer” refers to oligomeric compounds, primarily to oligonucleotides containing naturally occurring nucleotides such as adenine, guanine, cytosine, thymine and/or uracil, but may also include modified oligonucleotides (e.g., modified nucleotides, nucleosides, synthetic nucleotides having modified base moieties and/or modified sugar moieties (See, Protocols for Oligonucleotide Conjugates, Methods in Molecular Biology, Vol 26, (Sudhir Agrawal, Ed., Humana Press, Totowa, N.J., (1994)); and Oligonucleotides and Analogues, A Practical Approach (Fritz Eckstein, Ed., IRL Press, Oxford University Press, Oxford) that are able to prime polynucleotide (e.g., DNA) synthesis by an enzyme, typically in a template-dependent manner, i.e., the 3′ end of the primer provides a free 3′ —OH group to which further nucleotides are attached by the enzyme (e.g., DNA polymerase or reverse transcriptase) establishing a 3′ to 5′ phosphodiester linkage whereby nucleoside triphosphates are used and pyrophosphate is released. Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066. A review of synthesis methods is provided in Goodchild, 1990, Bioconjugate Chemistry 1(3):165-187.

A primer is typically a single-stranded deoxyribonucleic acid. The appropriate length of a primer depends on the intended use of the primer but typically ranges from 6 to 50 nucleotides. Short primer molecules (e.g., having a length within a range of 11-17 nucleotides) generally require cooler temperatures to form sufficiently stable hybrid complexes with a template (or target) nucleic acid.

As used herein, a “reagent” refers broadly to any agent used in a reaction, other than the analyte (e.g., nucleic acid molecule being analyzed). Illustrative reagents for a nucleic acid amplification reaction or sequencing assay include, but are not limited to, buffer, metal ions, polymerase, reverse transcriptase, primers, probes, template nucleic acid, nucleotides, labels, dyes, nucleases, adapters, oligo-coated beads, microparticles or droplets, and the like. Generally, reagents for enzymatic reactions include, for example, substrates, cofactors, buffers, metal ions, inhibitors, and/or activators.

As used herein, the term “sample” refers to a sample collected from a subject including, but not limited to, human and non-human animal subjects, that may be affected by or are suspected of infection by a pathogen (e.g., an infectious bacterium, protozoa, prion, fungi, algae, parasite or other microbe). The term also includes samples collected from the environment including, but not limited to, surface samples, water samples, soil samples and the like. A sample includes, but is not limited to, a cell, cell lysate, isolated DNA, isolated RNA, tissue section, tissue biopsy, liquid biopsy, blood, or other biological fluid (e.g., cerebrospinal fluid) obtained from a subject. A sample includes blood samples (e.g., whole peripheral blood, serum or plasma), tissue samples (e.g., fresh, frozen or Fixed Formalin Paraffin Embedded (FFPE) samples, biopsy samples (e.g., fine needle aspirates (FNAs)), excretions and secretions such as, saliva, sputum, urine, stool, plasma/serum, breast milk, sperm, semen, vaginal secretions, sweat, mucus, bile, and oral and genital mucosal swabs. The sample can include a clinical sample (e.g., a patient sample) for the purpose of diagnosis, detection, epidemiology, treatment, disease monitoring, and the like. In some instances, the sample comprises isolated RNA and/or DNA from a mammal (e.g., pig, cow, goat, sheep, rodent, rat, mouse, dog, cat, non-human primate or human). A tissue sample typically includes one or more cells obtained from a tissue of the subject or cells derived from a tissue obtained from the subject (e.g., cells in tissue culture). It will be apparent to one of ordinary skill in the art that a tissue sample can include cells obtained from a somatic tissue (e.g., liver, kidney, spleen, gall bladder, stomach, bladder, uterus, intestines, pancreas, colon, lung, heart, brain, muscle, bone, pharynx and larynx).

As used herein, the term “subject” refers to any member of the class animals, including, without limitation, humans and other primates, including non-human primates such as rhesus macaques, chimpanzees and other monkey and ape species; farm animals, such as cattle, sheep, pigs, goats and horses; domestic mammals, such as dogs and cats; laboratory animals, including rabbits, mice, rats and guinea pigs; birds and other reptiles, including domestic, wild, and game birds, such as chickens, turkeys, geese, ducks, lizards, alligators, and snakes; amphibians, including frogs, toads, salamanders, and newts; fish, such as salmon, and tilapia; and insects. The term does not denote a particular age or gender. Thus, adult, young, and newborn subjects are intended to be included as well as male and female subjects. In most instances, the subject is a host to the pathogen and the pathogen may rely on its ability to infect the host, for example the production of toxins, to enter cells and tissues within the host, and acquire host nutrients to maintain infectiousness. The term includes subjects who are experiencing or have experienced illness or disease associated with a particular taxon of pathogenic microorganisms or subjects who are infected (or suspected of being infected) with a particular taxon of pathogen but are not experiencing or demonstrating symptoms of illness or disease associated with the pathogen.

As used herein, a “target” refers to a molecule of interest to be detected in a sample. In some embodiments, the target is a nucleic acid molecule. In a one embodiment, the target is a target DNA, target RNA or target nucleic acid from a pathogen. In some embodiments, the target is a polynucleotide, such as dsDNA or ssDNA; RNA, such as ssRNA or dsRNA, or a DNA-RNA hybrid. In some embodiments, two or more target molecules are detected in a single sample. In some embodiments, the two or more target molecules may be related to each other (e.g., nucleic acids from the same taxon, genus or species of pathogens). In another embodiment, a first target molecule is from a first taxon of pathogenic microorganisms and a second target molecule is from a second taxon of pathogens. In some embodiments, the target nucleic can be from the host subject and not a pathogen.

In some instances, a target sequence or target nucleic acid molecule refers to a region, subsequence, or complete nucleic acid molecule which is to be amplified (e.g., RNA to cDNA, or amplification of DNA) or detected using the method, kits and compositions disclosed herein. Accordingly, amplification of one or more target sequences can include detection of one or more pathogenic microorganisms in a single sample, such as but not limited to, the detection and/or identification of a co-infection in the sample. For example, a clinical sample from a subject (e.g., a serum or urine sample from a human subject) can be evaluated for the presence (or absence) of an amplified target sequence present in the genome of a microorganism. Identification of two target sequences from distinct taxa from different domains (e.g., bacterial and fungal domains) would be indicative that the subject is infected by both pathogenic microorganisms (e.g., a fungal pathogen and a bacterial pathogen). Identification of the target sequence in the sample can be useful for the modulation of the form, dosage, or regime of treatment for the subject affected by the pathogen.

As used herein, the terms “treatment” and “treating” and the like, refer to methods or compositions for amelioration of disease or illness including any objective or subjective parameter such as abatement; remission; diminishing of symptoms or delaying the onset of symptoms; slowing in the rate of degeneration or decline; making the final point of degeneration less debilitating; and/or improving a subject's physical or mental well-being.

As used herein, the term “thermostable polymerase” refers to a polymerase enzyme that is heat stable, i.e., the enzyme catalyzes the formation of a primer extension product complementary to a template nucleic acid, and is not irreversibly denatured when subjected to elevated temperatures for the time needed to effect denaturation of double-stranded template nucleic acids (e.g., between 95° C.-99° C.). Thermostable polymerases have been isolated from Thermus flavus, T. ruber, T. thermophilus, T. aquaticus, T. lacteus, T. rubens, Bacillus stearothermophilus, and Methanothermus fervidus. Additionally, polymerases that are not thermostable can be employed in the PCR assays disclosed herein, for example by replenishing the polymerase between synthesis/extension and denaturation steps as it becomes denatured. Any polymerase or thermostable polymerase known in the art is suitable for use in the method disclosed herein.

The disclosure also provides embodiments directed to dehosting a sample prior to the identification of a taxon or taxa of pathogenic microorganisms in a sample. Such dehosting techniques and compositions relate to the selective cleavage of non-microbial nucleic acids in a sample containing both pathogen-based nucleic acids and non-pathogen-based nucleic acids (e.g., nucleic acids from a subject), so that the sample becomes greatly enriched with microbial nucleic acids. Examples of dehosting methods include those described in Feehery et al., PLoS ONE 8:e76096 (2013); Sachse et al., Journal of Clinical Microbiology 47:1050-1057 (2009); Barnes et al., PLoS ONE 9(10):e109061 (2014); Leichty et al., Genetics 198(2):473-81 (2014)); Hasan et al., J Clin Microbiol 54(4):919-27 (2016); and Liu et al., PLoS ONE 11(1):e0146064 (2016). Additionally, commercial kits for carrying out dehosting are also available, including the NEBNext Microbiome DNA Enrichment™ Kit, the Molzym MolYsis Basic™ kit, and MICROBEEnrich™ Kit.

In some embodiments, the dehosting methods and compositions disclosed herein takes advantage of properties associated with non-pathogen-based nucleic acids, including methylation at CpG residues, and associations with DNA-binding proteins, such as histones. For example, in a particular embodiment the dehosting methods and compositions can utilizes a nucleic acid binding protein that selectively binds with non-pathogen-based nucleic acids (e.g., histones, restriction enzymes). In a further embodiment, the dehosting methods and compositions can comprise a recombinant protein that selectively binds with non-pathogen-based nucleic acids, and which also selectively degrades non-pathogen-based nucleic acids, i.e., the recombinant protein comprises both a nonmicrobial nucleic acid binding domain and a nuclease domain. In a particular embodiment, the nucleic acid binding protein is a histone. Histones are found in the nuclei of eukaryotic cells, and in certain Archaea, namely Thermoproteales and Euryarchaea, but not in bacteria or viruses. In a further embodiment, histone bound non-pathogen-based nucleic acids can then be removed from the sample by use of a substrate which comprises an affinity agent that selectively binds to a histone protein, i.e., a histone-binding domain. Examples of affinity agents that can bind to a histone protein include, but are not limited to, chromodomain, Tudor, Malignant Brain Tumor (MBT), plant homeodomain (PHD), bromodomain, SANT, YEATS, Proline-Tryptophan-Tryptophan-Proline (PWWP), Bromo Adjacent Homology (BAH), Ankryin repeat, WD40 repeat, ATRX-DNMT3A-DNMT3L (ADD), or zn-CW. In another embodiment, the histone-binding domain can include a domain which specifically binds to a histone from a protein such as HAT1, CBP/P300, PCAF/GCNS, TIP60, HB01 (ScESA1, SpMST1), ScSAS3, ScSAS2 (SpMST2), ScRTT109, SirT2 (ScSir2), SUV39H1, SUV39H2, G9a, ESET/SETDB1, EuHMTase/GLP, CLL8, SpClr4, MLL1, MLL2, MLL3, MLL4, MLL5, SET1A, SET1B, ASH1, Sc/Sp SET1, SET2 (Sc/Sp SET2), NSD1, SYMD2, DOT1, Sc/Sp DOT1, Pr-SET 7/8, SUV4 20H1, SUV420H2, SpSet 9, EZH2, RIZ1, LSD1/BHC110, JHDM1a, JHDM1b, JHDM2a, JHDM2b, JMJD2A/JHDM3A, JMJD2B, JMJD2C/GASC1, JMJD2D, CARM1, PRMT4, PRMT5, Haspin, MSK1, MSK2, CKII, Mst1, Bmi/Ring1A, RNF20/RNF40, or ScFPR4, or a histone-binding fragment thereof.

In additional embodiment, the disclosure also provides for a nucleic acid binding protein or nucleic acid binding domain that selectively binds to DNA that comprises a methylated CpG. CG dinucleotide motifs (“CpG sites” or “CG sites”) are found in regions of DNA where a cytosine nucleotide is followed by a guanine nucleotide in the linear sequence of bases along its 5′ to 3′ direction. CpG islands (or CG islands) are regions with a high frequency of CpG sites. CpG is shorthand for 5′-C-phosphate-G-3′, that is, cytosine and guanine separated by one phosphate. Cytosines in CpG dinucleotides can be methylated to form 5-methylcytosine. Cytosine methylation occurs throughout the human genome at many CpG sites. Cytosine methylation at CG sites also occurs throughout the genomes of other eukaryotes. In mammals, for example, 70% to 80% of CpG cytosines may be methylated. In pathogenic microorganisms of interest, such as bacteria and viruses, this CpG methylation does not occur or is significantly lower than the CpG methylation in the human genome. Thus, dehosting can be achieved by selectively cleaving CpG methylated DNA.

In some embodiments, the disclosure provides for a dehosting method which comprises a nucleic acid binding protein or binding domain which binds to CpG islands or CpG sites. In another embodiment, the binding domain comprises a protein or fragment thereof that binds to methylated CpG islands. In yet another embodiment, the nucleic acid binding protein binding domain comprises a methyl-CpG-binding domain (MBD). An example of an MBD is a polypeptide of about 70 residues that folds into an alpha/beta sandwich structure comprising a layer of twisted beta sheet, backed by another layer formed by the alpha1 helix and a hairpin loop at the C terminus. These layers are both amphipathic, with the alpha1 helix and the beta sheet lying parallel and the hydrophobic faces tightly packed against each other. The beta sheet is composed of two long inner strands (beta2 and beta3) sandwiched by two shorter outer strands (beta1 and beta4). In a further embodiment, the nucleic acid binding protein or binding domain comprises a protein selected from the group consisting of MECP2, MBD1, MBD2, and MBD4, or a fragment thereof. In yet a further embodiment, the nucleic acid binding protein or binding domain comprises MBD2. In a certain embodiment, the nucleic acid binding protein or binding domain comprises a fragment of MBD2. In another embodiment, the nucleic acid binding protein or binding domain comprises MBD5, MBD6, SETDB1, SETDB2, TIP5/BAZ2A, or BAZ2B, or a fragment thereof. In yet another embodiment, the nucleic acid binding protein or binding domain comprises a CpG methylation or demethylation protein, or a fragment thereof. In a further embodiment, CpG bound nonmicrobial nucleic acids can then be removed from the sample by use of a substrate which comprises an affinity agent that selectively binds to a nucleic acid binding protein or binding domain which binds to CpG islands or CpG sites. Examples of affinity agents include antibodies or antibody fragments that selectively bind to a nucleic acid binding protein or binding domain which binds to CpG islands or CpG sites. Affinity agents comprising antibodies or antibody fragments can be bound to a substrate or alternatively may itself be bound by a second antibody which is bound to a substrate, thereby providing a means to separate and remove the nonmicrobial nucleic acids from a sample.

In another embodiment the disclosure provides for dehosting method that uses a nuclease, or a recombinant protein which comprises a nuclease domain, whereby the nuclease cleaves non-pathogen-based nucleic acids into fragments. In the latter case, the recombinant protein may also comprise a nucleic acid protein binding domain having activity for nucleic acid binding proteins (e.g., histones, methyl-CpG-binding proteins). The nuclease or nuclease can include, but are not limited to, a non-specific nuclease, an endonuclease, non-specific endonuclease, non-specific exonuclease, a homing endonuclease, and restriction endonuclease. In another embodiment, the nuclease domain is derived from any nuclease where the nuclease or nuclease domain does not itself have its own unique target. In yet another embodiment, the nuclease domain has activity when fused to other proteins. Examples of non-specific nucleases include FokI and I-TevI. In some embodiments, the nuclease domain is FokI or a fragment thereof. In a further embodiment, the nuclease domain is I-TevI or a fragment thereof. In yet a further embodiment, the FokI or I-TevI or fragment thereof is unmutated and/or wild-type. Further examples of nucleases include but are not limited to, Deoxyribonuclease I (DNase I), RecBCD endonuclease, T7 endonuclease, T4 endonuclease IV, Bal 31 endonuclease, endonucleaseI (endo I), Micrococcal nuclease, Endonuclease II (endo VI, exo III), Neurospora endonuclease, S1-nuclease, P1-nuclease, Mung bean nuclease I, Ustilago nuclease (Dnase I), AP endonuclease, and Endo R.

As used herein, “Polymerase Chain Reaction (PCR)” refers to a process in which one or more nucleic acid molecules are amplified typically through the use of one or more primers under suitable amplification conditions. PCR is described in U.S. Pat. Nos. 4,683,195; 4,683,202; and 4,965,188; Saiki et al., 1985, Science 230:1350-1354; Mullis et al., 1986, Cold Springs Harbor Symp. Quant. Biol. 51:263-273; and Mullis and Faloona, 1987, Methods Enzymol. 155:335-350. The development and application of PCR are described extensively in the literature. For example, a range of PCR-related topics are discussed in PCR Technology—principles and applications for DNA amplification, 1989, (ed. H. A. Erlich) Stockton Press, New York; PCR Protocols: A guide to methods and applications, 1990, (ed. M. A. Innis et al.) Academic Press, San Diego; and PCR Strategies, 1995, (ed. M. A. Innis et al.) Academic Press, San Diego. Commercial vendors, such as ThermoFisher Scientific (Waltham, Conn.) market PCR reagents and publish PCR protocols.

PCR typically employs two oligonucleotide primers, commonly referred to in the art as a primer pair (a forward and reverse primer) that hybridize to a template nucleic acid (e.g., DNA or RNA molecule). Primers useful in some embodiments of the disclosure include oligonucleotides capable of acting as points of initiation of nucleic acid synthesis of a pathogen's genome or expressed polynucleotides. Primers for PCR are typically single-stranded for maximum efficiency during amplification. Additionally, primers are often denatured, i.e., treated to promote linear, single-stranded primers in the amplification reaction. One method of denaturing primers is by heating (e.g., heating at 95° C. for 3-5 minutes).

If the template nucleic acid to be amplified is double-stranded, it is often needed to separate the two strands before it can be used as a template in PCR. Strand separation can be accomplished by any suitable denaturing methods known in the art including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions needed for denaturing template nucleic acids will depend, e.g., on the buffer salt concentration and the length and nucleotide composition of the nucleic acids being denatured, but typically ranges from about 90° C. to about 100° C. for a time depending on features of the reaction, such as but not limited to, melting temperature and nucleic acid length.

If the double-stranded template nucleic acid is denatured by heat, the reaction mixture is often allowed to cool to a temperature that promotes annealing of each primer to its target sequence. The temperature for annealing is usually from about 35° C. to about 65° C. (e.g., about 40° C. to about 60° C., about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase or reverse transcriptase is promoted or optimized, i.e., a temperature sufficient for nucleic acid extension to occur from the annealed primer to generate amplification products complementary to the template nucleic acid. The temperature should be sufficient to synthesize an extension/amplification product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; or about 60° C.). Extension times can be from about 10 sec to about 5 min (e.g., about 30 see to about 4 min; about 1 min to about 3 min; about 1 min 30 sec to about 2 min).

Since its inception, various amplification techniques have been described as variants or derivatives of PCR including, but not limited to, Ligase Chain Reaction (LCR, Wu and Wallace, 1989, Genomics 4:560-569 and Barany, 1991, Proc. Natl. Acad. Sci. USA 88:189-193); Polymerase Ligase Chain Reaction (Barany, 1991, PCR Methods and Applic. 1:5-16); Gap-LCR (PCT Patent Publication No. WO 90/01069); Repair Chain Reaction (European Patent Publication No. 439,182 A2), 3SR (Kwoh et al., 1989, Proc. Natl. Acad. Sci. USA 86:1173-1177; Guatelli et al., 1990, Proc. Natl. Acad. Sci. USA 87:1874-1878; PCT Patent Publication No. WO 92/0880A), NASBA (U.S. Pat. No. 5,130,238), Nested-Patch PCR (Varley and Mitra, (2008) Genome Research, 18:1844-50), asymmetric PCR (Wooddell & Burgess, (1996) Genome Research, 6:886-892), anchored PCR (Loh, (1991) Methods, 2, 1:11-19) inverse PCR (Ochman et al., (1988) Genetics, 120 (3):621-23), real-time quantitative PCR (Real Time-PCR) or quantitative PCR (qPCR) (Watson et al., (2004). Molecular Biology of the Gene (Fifth ed.). San Francisco: Benjamin Cummings), transcription based amplification system (TAS), strand displacement amplification (SDA), rolling circle amplification (RCA), hyper-branched RCA (HRCA) and Rapid Amplification of cDNA ends (RACE) (Lagarde et al., (2016), Nat. Comm., 7:1233. Additionally, digital PCR is a technique that allows quantitative measurement of the number of target molecules in a sample. The basic premise is to divide a large sample into a number of smaller subvolumes (partitioned volumes), whereby the subvolumes contain on average a low number or single copy of target. By counting the number of successful amplification reactions in the subvolumes, one can deduce the starting copy number of the target molecule in the starting volume (U.S. Pat. No. 8,722,334).

Methods to reduce non-specific hybridization and amplification of off-target sequences have been improved through the application of “hot-start” techniques. A hot-start method typically involves an initial high (e.g., 95° C.-100° C.) incubation temperature step, after which one or more important reagents for amplification are added to the reaction mixture (e.g., MgCl₂ or deoxyribonucleotides (dNTPs)). By raising the reaction mixture temperature prior to the introduction of at least one amplification reagent a reduction in self-forming secondary structures, reduction in non-specific cross-linking, and a reduction in primer dimers can be achieved. Another method of reducing the formation of non-specific amplification products relies on heat-reversible inhibition of DNA polymerase by DNA polymerase-specific antibodies, as described in U.S. Pat. No. 5,338,671. The antibodies are incubated with a DNA polymerase in a buffer at room temperature prior to the assembly of the reaction mixture in order to allow formation of the antibody-DNA polymerase complex. Antibody inhibition of the DNA polymerase activity is inactivated by a high temperature incubation step prior to amplification.

Each cycle of PCR typically comprises three steps: denaturation, annealing, and synthesis; the method frequently involves about 15 to about 30 cycles and is routinely automated using a thermocycler. The steps of denaturation, annealing, and synthesis can be repeated as often as needed to produce the desired quantity of amplification products (e.g., corresponding to a required amount of target molecules). Often, the limiting factors in the amplification reaction are the amounts of primers, thermostable enzyme(s), and nucleoside triphosphates present in the reaction. The cycling steps (i.e., denaturation, annealing, and extension) are typically repeated at least once. The number of cycling steps will depend on the nature of the sample and/or the frequency of the target molecules in the sample. If the target molecule (e.g., Zika virus genome copies or other pathogen genome) is present in low numbers in a complex mixture of nucleic acids (e.g., a blood sample from a host), more cycling steps may be required to amplify the target molecule to a point where the amount of amplified product is sufficient for detection by the method.

PCR allows for rapid and specific diagnosis of infectious diseases, infectious organisms or microbes, including those caused by bacteria, fungi, protazoa etc. PCR also permits identification of non-cultivatable or slow-growing microorganisms such as mycobacteria, anaerobic bacteria, viruses from tissue culture assays or animal models. Multiplex PCR (a set of primer that allow amplification of at least two targets (e.g., amplification of at least 2 different genes or sub-regions thereof) provides additional flexibility to detect multiple target pathogenic microorganisms in a single assay or reaction. Other applications of PCR include detection of infectious pathogenic microorganisms and the discrimination of non-pathogenic from pathogenic strains (Salis A., (2009). Applications in Clinical Microbiology. Real-Time PCR: Current Technology and Applications). Amplification products from PCR reactions can be identified via gel electrophoresis although typically most assays utilize real-time PCR, where the amplification product of the PCR reaction is monitored in each cycle of amplification (i.e., in real-time) through the use of a double-stranded fluorescent dye or labeled probe. For example, PCR in veterinary applications can be used to detect bacterial pathogenic microorganisms including, but not limited to, Brachyspiraspp, Chlamydophila abortus, Chlamydophila psittaci, Coxiella burnetii, avian Coxiella-like organism, Lawsonia intracellularis, Mycobacterium avium subsp paratuberculosis, different species of Mycoplasma, and Streptococcus equi subsp equi. Identification of pathogenic microorganisms across mammalian species is useful when addressing zoonotic or potentially zoonotic infections.

Nucleic acid amplification of the target molecule can be carried out using any suitable amplification method, such as, but not limited to, PCR and related methods. In particular embodiments, amplification of a portion of a gene or genomic region from a pathogen present in a sample can be performed by real-time amplification, such as real-time PCR or reverse transcription PCR (RT-PCR). DNA sequencing can also be carried out using any of the various DNA sequencing methods and sequencing platforms available in the art, such as, but not limited to Illumina Inc., Oxford Nanopore Technologies, Inc., Ion Torrent, Helicos Biosciences Corp., Fluidigm, Nimblegen, Roche Sequencing, and the like. Exemplary DNA sequencing methods are described in the Examples section.

As used herein, a “sequencing assay” refers to a method for determining the order of nucleotides in at least a part of a nucleic acid molecule. A well-known method of sequencing is the “chain termination” method first described by Sanger et al., PNAS (USA) 74(12): 5463-5467 (1977) and detailed in SEQUENAS™ 2.0 product literature (Amersham Life Sciences, Cleveland) and in European Patent EP-B1-655506. In essence, DNA to be sequenced is obtained (e.g., isolated from a cell or sample), rendered single stranded (denatured), and placed into four vessels. Each vessel contains components to amplify the DNA, which include a template-dependent DNA polymerase, a primer complementary to the initiation site of sequencing of the DNA to be sequenced and deoxyribonucleotide triphosphates for each of the bases A, C, G and T, in a buffer conducive for hybridization between the primer and the DNA to be sequenced and chain extension of the hybridized primer. In addition, each of the vessels contains a small quantity of one type of dideoxynucleotide triphosphate, e.g. dideoxyadenosine triphosphate (“ddA”), dideoxyguanosine triphosphate (“ddG”), dideoxycytosine triphosphate (“ddC”), dideoxythymidine triphosphate (“ddT”). In each vessel, the target DNA is denatured and hybridized with a primer. The primers are extended to form a primer extension product that is complementary to the target DNA (i.e., the template nucleic acid). When a dideoxynucleotide is incorporated into the extending polymer, the polymer is prevented from further extension (blocked). Accordingly, in each vessel, a set of extended polymers of specific lengths are formed which are indicative of the positions of the nucleotide corresponding to the dideoxynucleotide in that vessel. The extended primer products are evaluated, for example using gel electrophoresis, to determine the sequence of the new polymeric strands.

More recently, the Sanger technique has been surpassed by Next-Generation Sequencing (NGS) platforms. The NGS platforms include automated, massively parallel, high-throughput sequencing methods (see, for example, Illumina iSeq, HiSeq, MiSeq, & NextSeq, Ion Torrent PGM and Proton, Roche 454 Life Sciences, Applied Biosystems SOLiD, Oxford Nanopore Technologies MinION, GridION, and PromethION instruments, and other DNA sequencing platforms). Some of the NGS methods include labels for detection of target molecules (e.g., one, two, three, four, or all nucleotide types corresponding to incorporation of A, G, T, or C, are labeled). In other embodiments, one, two, three, or all nucleotide types are label-free (See, ion semiconductor sequencing, such as the Ion Torrent and DNAe sequencing platforms) such that polymerization or nucleotide incorporation is measured by hydrogen ion release, pyrophosphate release, or a combination thereof. Other examples of NGS techniques contemplated for use with the disclosure include metagenomic NGS, which typically includes “shotgun” based amplification of one or more regions of a target nucleic acid molecule, such as but not limited to bacterial or viral genomes. Typically, metagenomic sequencing involves analysis of genetic information obtained from a sample that contains a plurality of microorganisms, including uncultured organisms. Generally, metagenomic sampling involves sample collection, isolation of nucleic acid molecules of interest, DNA sequencing of the nucleic acid molecules of interest to obtain sequencing reads, alignment of the sequencing reads to a reference genome, and identification of nucleic acid molecules having a sequence similarity above a certain threshold to one or more microorganisms.

In one embodiment, NGS methods of particular interest include a library preparation and/or a sequencing library. For example, a sample can contain an RNA target of interest. The sample may be treated with a DNA destroying reagent (e.g., DNase) to isolate RNA molecules of interest. The RNA molecules can be amplified using primers and any amplification method in the art (e.g., reverse transcriptase) to form cDNA molecules and optionally, first- and second-strand DNA synthesis based on the cDNA molecules to increase the amount of DNA molecules in the reaction, thereby forming a library preparation. In some instances, the library preparation can be further amplified using the same or preferentially, different primers to generate increased amounts of the amplified DNA molecules from the library preparation, thereby forming a sequencing library. The sequencing library (or the library preparation may be used with any appropriate sequencing platform and corresponding sequencing assay (e.g., input DNA applied to the sequencing platform, such as Illumina HiSeq).

Metagenomic next-generation sequencing (mNGS) is a promising candidate approach for broad-spectrum pathogen identification in clinical samples as nearly all potential pathogenic microorganisms—viruses, bacteria, fungi, and parasites—can be detected on the basis of uniquely identifying DNA and/or RNA shotgun sequences. This method has been successfully applied for clinical diagnosis of infectious diseases, outbreak surveillance by whole-genome viral sequencing, and pathogen discovery. Thus, mNGS can be a particularly useful diagnostic tool for addressing unknown outbreaks, as it does not require a priori targeting of pathogenic microorganisms that may suddenly emerge in a new geographic region. However, current issues related to cost, sequencing depth, and background contamination limit the accuracy of mNGS-based diagnostics relative to specific PCR testing.

Described herein are methods, compositions, and kits for detecting the presence (or absence) of a particular taxon of pathogens, such as but not limited to, a bacterium, fungi, protazoa etc. in a sample. These methods are useful in the areas of diagnosis of pathogenic infections, epidemiology, and disease surveillance, among others.

The disclosure provides a number of universal primers that comprise, consist essential of, or consist of the sequences as set forth in Table 1 for the detection of various microbial populations as set forth in Table 1. The primers of Table 1 can comprise 1-5 additional nucleotides at either end or 1-5 fewer nucleotides in some instances.

TABLE 1
UNIVERSAL BACTERIAL PRIMERS

| | |
|---|---|
| 302F-universal-Tm54 | CACACTGGRACTGAGAYACGG (SEQ ID NO: 1) |
| 333F-universal-Tm55-2X | ACTCCTACGGGAGGCWGCA (SEQ ID NO: 2) |
| 528F-universal-Tm57-4X | GTGCCAGCAGYYGCGGTA (SEQ ID NO: 3) |
| 802R-universal-Tm52-8X | GAYTACYRGGGTATCTAATCC (SEQ ID NO: 4) |
| 897R-universal-Tm53-6X | CCCCGTCAATTHMTTTGAGTTT (SEQ ID NO: 5) |
| 1072R-universal-Tm55-2X | CGTTRCGGGACTTAACCCAACA (SEQ ID NO: 6) |
| 1166R-Tm53-4X | TCRTCCYCACCTTCCTCC (SEQ ID NO: 7) |
| 1380R-Tm52-8X | YCCGRGAACGTATTCACSG (SEQ ID NO: 8) |

BABESIA PRIMERS

| | |
|---|---|
| 18S-304F-2-fold-Tm50.7 | GGTATTGGCCTACCGRG (SEQ ID NO: 9) |
| 18S-413F-0-fold-Tm51.1 | TACCCAATCCTGACACAGG (SEQ ID NO: 10) |
| 18S-877R-2-fold-Tm53.4 | GCTTTCGCAGTRGTTCGTCTT (SEQ ID NO: 11) |
| 18S-931R-0-fold-Tm51.8 | CGTCTTCGATCCCCTAACTT (SEQ ID NO: 12) |
| 18S-1018F-0-fold-Tm51.1 | GACTCCTTCAGCACCTTGA (SEQ ID NO: 13) |
| 18S-1619R-0-fold-Tm50.5 | CGAATAATTCACCGGATCACT (SEQ ID NO: 14) |
| 18S-1679R-0-fold-Tm51.7 | AGTTTTGTGAACCTTATCACTTAAAG (SEQ ID NO: 15) |

MYCOBACTERIAL PRIMERS

| | |
|---|---|
| Mycobacterium-rpoB-259F-4XTm47.1-51.9 | CACGGCAAYAAGGGYGT (SEQ ID NO: 16) |
| Mycobacterium-rpoB-274F-6XTm45.8-50.3 | GTBATCGGCAAGATYCTC (SEQ ID NO: 17) |
| Mycobacterium-rpoB-697R-1XTm51.9 | TCACCGGGTACGGGAAC (SEQ ID NO: 18) |
| Mycobacterium-rpoB-755R-4XTm47.1-49.5 | GCGTGRATCTTGTCRTC (SEQ ID NO: 19) |
| Mycobacterium-hsp65-322F-2XTm = 52.6 | TACGAGAAGATCGGCGCY (SEQ ID NO: 20) |
| Mycobacterium-hsp65-282F-1XTm = 52.6 | GGTGTGTCCATCGCCAAG (SEQ ID NO: 21) |
| Mycobacterium-hsp65-650R-3XTm = 51.9 | CTCGTTGCCVACCTTGTC (SEQ ID NO: 22) |
| Mycobacterium-hsp65-670R-2XTm = 52.6 | CTCGACGGTGATGACRCC (SEQ ID NO: 23) |

FUNGAL PRIMERS

| | |
|---|---|
| Fungal-18S-SSU-forward1-4X | GTACACACKCCYGTCG (SEQ ID NO: 24) |
| Fungal-18S-SSU-forward2-6X | TGYAATTDTTGCTCTTCAACGAG (SEQ ID NO: 25) |
| Fungal-296R-4X | GCTSCGTTCTTCATCGATSC (SEQ ID NO: 26) |
| Fungal-350R-4X | GTTCAAGAYTCRATGATTCAC (SEQ ID NO: 27) |
| Fungal-296R-Pneumocystis | GCCACGTTCTTCATCGACGC (SEQ ID NO: 28) |
| Fungal-350R-Pneumocystis | GTTCAAAAATTCGATGATTCAC (SEQ ID NO: 29) |

R = A or G; Y = C or T; W = A or T; K = G or T; M = A or C; B = C or G or T; H = A or C or T; V = A or C or G; S = G or C; D = A, G or T
Tm = melting temperature in Celsius
F = forward primer
R = reverse primer
1X, 2X, 4X, 6X, 8X refer to degeneracy of the primer and input concentration must account for the degeneracy (i.e., 2X degenerate primer is added at 2X the concentration of a 1X primer (non-degenerate) etc.)

It should be recognized that any of the sequence of Table 1 can have T replaced by U for RNA.

The universal primers of Table 1 are useful for identification and/or amplification of nucleic acid associated with the microbial class for which they are directed. For example, if one of skill in the art wanted to determine the presence of a fungal organism in a sample, primers having SEQ ID NOs: 24-29 would be used to identify and/or amplify nucleic acids from the sample using the methods described above (e.g., PCR or other amplification techniques). Thus, determining the presence of a fungal organism being present in the sample. Further sequencing could be used to determine a specific taxa (e.g. species) of the fungal organism, thus identifying, for example, an infection by or the presence of a fungal organism.

The primers of Table 1 are also useful as a trap for total microbial-derived target material (e.g., nucleic acids). Such trapped material may then be sequenced, or cloned and sequenced and/or subjected to primer/probe interrogation. Consequently, the disclosure provides an ability to detect microbes from samples which are difficult to cultivate and that would in all practicality remain undetected or under-estimated by viable culture count methods or, alternatively, microbes that are in an aggregated or coaggregated state or contaminated, such as in mixed samples. In addition, the application of the universal primers of the disclosure enable rapid identification and/or differentiation of microbes in, for example, infections. This is particularly useful, for example, in assessing modes of treatment.

The compositions and methods of the disclosure are applicable to a range of industries including the medical, agricultural and industrial industries with specific uses including enviroprotection, bioremediation, medical diagnosis, water quality control or food quality control.

The disclosure generally relates to methods for detecting a taxon of pathogenic microorganisms in a sample, wherein the sample may also contain host DNA and/or one or more additional and different taxon of pathogens. In one embodiment, the disclosure generally relates to a method of detecting a particular taxon of pathogenic microorganisms in a sample, comprising, (a) obtaining a sample (from the environment or a subject) to be screened for a particular taxon of pathogens; (b) applying a sequencing assay to the sample to obtain sequence reads, the sequencing assay including primers having lengths that are within a range of 11 bp to 25 bp, wherein the primers were identified to be suitable to identify organisms in a particular taxa; (c) aligning a first portion of the sequencing reads to a first reference genome for the particular taxon of pathogens; (d) aligning a second portion of the sequencing reads to a second reference genome corresponding to a different taxon of pathogens; and (e) determining whether the particular taxon and/or the different taxon of pathogenic microorganisms is present in the sample based on the alignment of the first and second portion of the sequencing reads.

The sample analyzed by the methods provided herein can be any sample including, but not limited to, any type of clinical sample or any type of environmental sample. In some embodiments, the sample contains a cell, tissue, or a bodily fluid. In some embodiments, the sample is a liquid or fluid sample. In some embodiments, the sample contains a body fluid such as whole blood, plasma, serum, urine, stool, saliva, lymph, spinal fluid, synovial fluid, nasal swab, respiratory secretions, vaginal fluid, amniotic fluid, or semen. In some embodiments, the sample comprises cells or tissue. In some embodiments, cells, cell fragments, or exosomes are removed from the sample, such as by centrifugation or filtration. In some embodiments, the sample is a biological sample. In some embodiments, the sample may be an unprocessed sample (e.g., whole blood) or a processed sample (e.g., serum, plasma) that contains cell-free or cell-associated nucleic acids. In some embodiments, the sample is enriched for certain types of nucleic acids, e.g., DNA, RNA, cell-free DNA, cell-free RNA, cell-free circulating DNA, cell-free circulating RNA, etc. In one embodiment, the sample is processed to isolate nucleic acids or to separate nucleic acids from other cellular components or nucleic acids within the sample (e.g., DNA or RNA isolation). In some embodiments, the sample is enriched for pathogen- or microbial-specific nucleic acids. In another embodiment, the sample comprises RNA or DNA from a subject infected with, or suspected of harboring an infectious pathogen.

In one embodiment, the sample comprises target nucleic acids. The target nucleic acids refer to nucleic acids to be analyzed in the sample. In some embodiments, the target nucleic acids are cell-free nucleic acids. For example, the target nucleic acids may be cell-free DNA, cell-free RNA (e.g., cell-free mRNA, cell-free miRNA, cell-free siRNA), or any combination thereof. In certain cases, the cell-free nucleic acids are pathogen nucleic acids, e.g., nucleic acids from pathogenic microorganisms such as bacteria, fungi, algae, and eukaryotic parasites. In some embodiments, different types of nucleic acids are present in the sample at the same time (e.g., host DNA or RNA and pathogen DNA or RNA).

In some embodiments, the sample is from a human subject, such as a human patient. In some embodiments, the sample may also be from any other type of subject including any plant, mammal, non-human mammal, non-human primate, domesticated animal (e.g., laboratory animals, household pets, or livestock), or non-domesticated animal (e.g., wildlife). In some embodiments, the subject is a dog, cat, rodent, mouse, hamster, cow, bird, chicken, pig, horse, goat, sheep, rabbit, or monkey. In some embodiments, the sample is from an environment (e.g., a water source, soil, food source, household or office or hospital items) and the like.

In one embodiment, the sample contains a certain amount, titer or concentration of target nucleic acids. Target nucleic acids within a sample may include double-stranded (ds) nucleic acids, single stranded (ss) nucleic acids, DNA, RNA, cDNA, dsDNA, ssDNA, circulating nucleic acids, circulating cell-free nucleic acids, circulating DNA, circulating RNA, genomic DNA, exosomes, cell-free pathogen nucleic acids, circulating pathogen nucleic acids, or any combination thereof. For example, circulating cell-free nucleic acids includes cell-free nucleic acids circulating in the bloodstream of the subject.

The sample may be obtained by any means known in the art. For example, the sample may be obtained by syringe (such as a FNA), blood draw, or direct placement into a vessel (such as urine, semen, feces, sputum, etc.), by swab, aspiration and the like. In some embodiments, obtaining the sample can include one or more processes that refine, purify and/or isolate the sample from its original composition, such as, but not limited to, nucleic acid extraction kits.

In one embodiment, the subject is a host organism (e.g., a human) infected with a pathogen, at risk of infection by a pathogen, or suspected of having a pathogenic infection. In some embodiments, the subject is suspected of having a particular infection, e.g., suspected of exposure to a bacterial pathogen etc. In other embodiments, the subject is suspected of having an infection of unknown origin. In some embodiments, a host is infected with more than one pathogen (e.g., a bacterial infection and co-infection with a virus, fungi or parasite). In some embodiments, a subject has been diagnosed with, or is at risk for developing symptoms associated with bacterial or fungal infection. In some embodiments, the subject is healthy and the methods disclosed herein are used to confirm the absence of a pathogen in the subject. In some embodiments, the subject is susceptible or is at risk of a pathogenic infection (e.g., an immunocompromised patient, elderly patient, newborn infant, is situated or has recently visited a locale known to possess infected subjects). In one example, the subject from whom the sample is obtained includes a mammalian host. In a specific embodiment, the subject includes a human host.

In some embodiments, the methods (and associated compositions and kits) disclosed herein are useful for detecting the presence of a first taxon of pathogenic microorganisms present in a sample. In another embodiment, the methods (and associated compositions and kits) disclosed herein are useful for detecting the absence of a particular taxon of pathogenic microorganisms present in a sample. The methods allow for the detection of one or more pathogenic microorganisms in a sample using a set of primers. In one embodiment, the method includes detection of 2, 3, 4, 5, 6, 7, 8, 9, 10 or more, pathogenic microorganisms from a single sample. In another embodiment, the method includes detection of at least two different taxa of pathogenic microorganisms from a single sample, e.g., a sample from a human subject. In some embodiments, the method includes determining whether a first taxon of pathogenic microorganisms is present in the sample (for example, based on alignment of one or more amplified nucleic acids obtained during the sequencing assay against a reference genome of the first taxon of pathogens). In another embodiment, the method includes determining whether a first taxon of pathogen is absent from the sample (for example, based on alignment of one or more amplified nucleic acids obtained during the sequencing assay against a reference genome of the first taxon of pathogens).

The methods provided herein (and associated kits and compositions) can be used to detect a plurality of pathogenic microorganisms present in a single sample. In one embodiment, the method includes detecting at least one bacterial taxon in the sample. In another embodiment, the method includes detecting at least one fungal taxon and one bacterial taxon in the sample (i.e., a co-infection). In yet another embodiment, the method includes detecting at least one bacterial and/or at least one fungal, and a parasitic infection in the sample (i.e., a co-infection). In one embodiment, the method includes detecting at least one bacterial pathogen in a sample.

In another embodiment, the method provides for the detection of one of more fungal genera. An exemplary list of fungal genera is provided in List 1. It will be apparent to one of ordinary skill in the art that the fungal genera provided in List 1 is not to be construed as exhaustive. In yet another embodiment, the method provides for the detection of one of more bacterial genera. An exemplary list of bacterial genera is provided in List 3. It will be apparent to one of ordinary skill in the art, that the bacterial genera provided in List 3 is not to be construed as exhaustive.

In some embodiments, one or more other taxa of pathogen are identified that are distinct from the first taxon of pathogenic microorganisms against which the sample is screened. The sample can be screened for a plurality of pathogen taxa, although the first taxon of pathogenic microorganisms is typically present in the sample at a lower titer than the one or more other taxa of pathogens. In one embodiment, the one or more other taxa of pathogenic microorganisms includes a bacterial, fungal, algal, protozoan, and/or microscopic parasite. In one example, the one or more other taxa of pathogenic microorganisms is selected from any of the genera provided in List 1 and List 3.

The methods (and associated kits and compositions) provided herein can be used to detect a taxon of pathogenic microorganisms in a sample from a subject (e.g., target nucleic acids) via a sequencing assay such as, multiplex RT-qPCR. The target nucleic acids can include, but are not limited to, whole or partial genomes, genetic loci, genes, exons, or introns. In one embodiment, the methods provided herein detect pathogenic target nucleic acids from a biological sample obtained from a subject. In some cases, the pathogenic target nucleic acids are present in complex clinical sample (e.g., an unprocessed sample such as whole blood or a processed sample such as serum) containing nucleic acids from the subject (i.e., the host) and the pathogen. In some embodiments, the pathogenic target nucleic acids are associated with an infectious disease. In another embodiment, the pathogen target nucleic acids are bacterial nucleic acids.

In some embodiments, the pathogen nucleic acids are present in a tissue sample, such as a tissue sample from a site of infection. In other embodiments, the pathogen nucleic acids have migrated from the site of infection; for example, it may be obtained from a sample containing circulating cell-free nucleic acids (e.g., circulating cf-DNA or cf-RNA).

In some embodiments, the target nucleic acids may make up a very small portion of the entire sample under evaluation, e.g., less than 1%, less than 0.5%, less than 0.1%, less than 0.01%, less than 0.001%, less than 0.0001%, less than 0.00001%, less than 0.000001%, or less than 0.0000001% of the total nucleic acids in the sample. In another embodiment, the target nucleic acids may make up from about 0.00001% to about 0.5% of the total nucleic acids in a sample. Often, the total nucleic acids in a sample may vary. For example, total cell-free nucleic acids (e.g., DNA or RNA) may be in a range of 1-100 ng/ml, e.g., (about 1, 5, 10, 20, 30, 40, 50, 80, 100 ng/ml). In some cases, the total concentration of cell-free nucleic acids in a sample is outside of this range (e.g., less than 1 ng/ml; in other cases, the total concentration is greater than 100 ng/ml). In another embodiment, total DNA in a sample (e.g., genomic, mitochondrial and pathogenic DNA extracted and purified from 100 μl of whole blood) may be in excess of 3 μg (see, Qiagen Dneasy Blood and Tissue purification kit, Catalog No. 69504). In some embodiments, the sample may contain a low viral titer of pathogen target nucleic acids which would still be elevated as compared to a non-infected, healthy sample. For example, pathogen target nucleic acids may make up less than 0.001% of total nucleic acids in an infected sample.

The length of target nucleic acids can vary. In some cases, target nucleic acids may be about or at least about 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000 or more nucleotides (or base pairs) in length, or a range of lengths between or including any two of the forgoing values (e.g., from about 30 to about 600 base pairs or nucleotides in length, from about 30 to about 250 base pairs or nucleotides in length, etc.). In some embodiments, the target nucleic acids are relatively short, e.g., less than 600 base pairs or nucleotides in length. In yet another embodiment, the target nucleic acids may be between 30 and 150 base pairs or nucleotides in length.

In some embodiments, the target nucleic acids include, but are not limited to, double-stranded (ds) nucleic acids, single stranded (ss) nucleic acids, DNA, RNA, cDNA, dsDNA, ssDNA, circulating nucleic acids, circulating cell-free nucleic acids, circulating DNA, circulating RNA, cell-free nucleic acids, cell-free DNA, cell-free RNA, circulating cell-free DNA, cell-free dsDNA, cell-free ssDNA, circulating cell-free RNA, genomic DNA, cell-free pathogen nucleic acids, circulating pathogen nucleic acids, circular DNA, circular RNA, circular single-stranded DNA, circular double-stranded DNA, or any combination thereof. The target nucleic acids are preferably nucleic acids derived from pathogenic microorganisms including but not limited to bacteria, fungi, parasites and other infectious microbes, including eukaryotic parasites. In some embodiments, target nucleic acids may be from the subject (e.g., host) as opposed to, or in addition to, target nucleic acids from a taxon of pathogens.

The methods (and associated compositions and kits) disclosed herein provide improved identification and/or quantification of target nucleic acid molecules in a sample from a subject, e.g., by RT-qPCR and/or NGS, particularly when the target nucleic acid molecules are present in low abundance in the sample (e.g., low viral titer) or when multiple pathogenic microorganisms are present. Additionally, the methods provided herein can be used to increase the yield of the target, particularly when the starting sample has relatively low amounts of the target.

In some embodiments, a microbe selected from the group consisting of bacteria, mycobacteria, babesia, fungi and any combination thereof can be detected by using the compositions of disclosure in a lateral flow assay based device. Lateral flow assay (LFA) based devices are among very rapidly growing strategies for qualitative and quantitative analysis. Lateral flow assays are performed over a strip, different parts of which are assembled on a plastic backing. These parts include a sample application pad, a conjugate pad, a nitrocellulose membrane and an adsorption pad. The nitrocellulose membrane is further divided into test and control lines. Pre-immobilized reagents at different parts of the strip become active upon flow of liquid sample. Lateral flow assays combine unique advantages of biorecognition probes and chromatography. Lateral flow assays basically combine a number of variants such as formats, biorecognition molecules, labels, detection systems and application.

Strips used for lateral flow assays contain four main components: a sample application pad, a conjugate pad, nitrocellulose membranes, and an adsorbent pad.

Sample application pad: The sample application pad is made of cellulose and/or glass fiber. The sample is applied on this pad to start the assay. Its function is to transport the sample to other components of lateral flow test strip (LFTS). The sample pad should be capable of transportation of the sample in a smooth, continuous and homogenous manner. The sample application pads are sometimes designed to pretreat the sample before its transportation. This pretreatment may include separation of sample components, removal of interfering agents, adjustment of pH, etc.

Conjugate pad: The conjugate pad is the place where labeled bio recognition molecules are dispensed. The material of conjugate pad should immediately release labeled conjugate upon contact with moving liquid sample. The labeled conjugate should stay stable over entire life span of the lateral flow strip. Any variations in dispensing, drying or release of conjugate can change results of the assay significantly. Poor preparation of labeled conjugate can adversely affect sensitivity of assay. Glass fiber, cellulose, poly-esters and some other materials are used to make conjugate pad for the lateral flow assay. The nature of the conjugate pad material has an effect on release of labeled conjugate and sensitivity of assay.

Nitrocellulose membrane: The Nitrocellulose membrane is highly important in determining sensitivity of the lateral flow assay. Nitrocellulose membranes are available in different grades. Test and control lines are drawn over this piece of membrane. An ideal membrane should provide support and good binding to capture probes (antibodies, aptamers etc.). Nonspecific adsorption over test and control lines may affect results of the assay significantly, thus a good membrane will be characterized by lesser non-specific adsorption in the regions of test and control lines. Wicking rate of nitrocellulose membrane can influence assay sensitivity. These membranes are easy to use, inexpensive, and offer high affinity for proteins and other biomolecules. Proper dispensing of bioreagents, drying and blocking play a role in improving sensitivity of assay.

Adsorbent pad: The adsorbent pad works as sink at the end of the strip. It also helps in maintaining the flow rate of the liquid over the membrane and stops back flow of the sample. The adsorbent capacity to hold liquid can play an important role in results of assay. All these components are fixed or mounted over a backing card.

Various formats can be adopted into the lateral flow assay, including the sandwich format, the competitive format and the multiplex detection format.

Sandwich Format: In a typical sandwich format, label (enzymes or nanoparticles or fluorescent dyes) coated antibody or aptamer is immobilized at the conjugate pad. This is a temporary adsorption which can be flushed away by flow of any buffer solution. A primary antibody or aptamer against target analyte is immobilized over test line. A secondary antibody or probe against labeled conjugate antibody/aptamer is immobilized at control zone. Sample containing the analyte is applied to the sample application pad and it subsequently migrates to the other parts of strip. At the conjugate pad, target analyte is captured by the immobilized labeled antibody or aptamer conjugate and results in the formation of labeled antibody conjugate/analyte complex. This complex now reaches the nitrocellulose membrane and moves under capillary action. At the test line, labeled antibody conjugate/analyte complex is captured by another antibody which is primary to the analyte. The analyte becomes sandwiched between the labeled and primary antibodies forming a labeled antibody conjugate/analyte/primary antibody complex. Excess labeled antibody conjugate will be captured at a control zone by a secondary antibody. Buffer or excess solution goes to absorption pad. The intensity of color at the test line corresponds to the amount of target analyte and is measured with an optical strip reader or visually inspected. Appearance of color at control line ensures that a strip is functioning properly.

Competitive format: A competitive format suits best for low molecular weight compounds which cannot bind two antibodies simultaneously. Absence of color at test line is an indication for the presence of analyte while appearance of color both at test and control lines indicates a negative result. The competitive format has two layouts. In the first layout, solution containing target analyte is applied onto the sample application pad and prefixed labeled biomolecule (antibody/aptamer) conjugate gets hydrated and starts flowing with the moving liquid. The test line contains pre-immobilized antigen (same analyte to be detected) which binds specifically to label conjugate. Control line contains pre-immobilized secondary antibody which has the ability to bind with labeled antibody conjugate. When liquid sample reaches at the test line, pre-immobilized antigen will bind to the labeled conjugate in case target analyte in sample solution is absent or present in such a low quantity that some sites of labeled antibody conjugate were vacant. Antigen in the sample solution and the one which is immobilized at test line of strip compete to bind with labeled conjugate. In another layout, labeled analyte conjugate is dispensed at conjugate pad while a primary antibody to analyte is dispensed at the test line. After application of analyte solution, a competition takes place between analyte and labeled analyte to bind with primary antibody at test line.

Multiplex detection: Multiplex detection format is used for detection of more than one target species and assay is performed over the strip containing test lines equal to number of target species to be analyzed. It is highly desirable to analyze multiple analytes simultaneously under same set of conditions. Multiplex detection format is very useful in clinical diagnosis where multiple analytes which are inter-dependent in deciding about the stage of a disease are to be detected. Lateral flow strips for this purpose can be built in various ways, i.e., by increasing length and test lines on conventional strip, making other structures like stars or T-shapes.

Various biorecognition molecules can be used with the lateral flow assay, including antibodies, aptamers, and molecular beacons.

Antibodies: Antibodies are employed as biorecognition molecules on the test and control lines of lateral flow strip and they bind to target analyte through immunochemical interactions. Resulting assay is known as lateral flow immunochromatographic assay (LFIA). Antibodies are available against common contaminants but they can also be synthesized against specific target analytes. An antibody which specifically binds to a certain target analyte is known as primary antibody but the one which is used to bind a target containing designs, formats and applications of lateral flow assay antibody or another antibody is known as secondary antibody.

Aptamers: Aptamers are the artificial nucleic acids and their discovery was reported by two groups in 1990. Aptamers have very high association constants and can bind selectively with a variety of target analytes. Organic molecules having molecular weights in the range of 100-10,000 Da are outstanding targets for aptamers. Because of their unique affinity toward target molecules, very closely related interferences can be differentiated. They are preferred over antibodies due to many features which include easy production process, simple labeling process, amplification after selection, straightforward structure modifications, unmatched stability, reproducibility and versatility of closely located quencher.

Molecular beacons: Molecular beacons can bind with high specificity and selectivity to nucleic acid sequences, toxins, proteins and other target molecules. Molecular beacons are composed of 15-30 base pairs in loop which are complimentary to target analyte and 4-6 base pairs at double stranded stem. Molecular beacons are being used in messenger RNA detection, intercellular imaging, protein and small molecule analysis, biosensors, biochip development, single nucleotide polymorphism and gene expression studies.

The list of materials that can be used as a label in a lateral flow assay is extensive and includes gold nanoparticles, colored latex beads, magnetic particles, carbon nanoparticles, selenium nanoparticles, silver nanoparticles, quantum dots, up converting phosphors, organic fluorophores, textile dyes, enzymes, liposomes and others. Any material that is used as a label should be detectable at very low concentrations and it should retain its properties upon conjugation with biorecognition molecules. This conjugation is also expected not to change the features of the bio-recognition probes. The ease in conjugation with biomolecules and stability over longer period of time are desirable features for a good label. Concentrations of labels down to 10⁻⁹ M are optically detectable. After the completion of assay, some labels generate direct signals (as color from gold colloidal) while others require additional steps to produce analytical signals (as enzymes produce detectable product upon reaction with suit-able substrate). Hence the labels which give direct signal are preferable in LFA because of less time consumption and reduced procedure.

Colloidal gold nanoparticles are the most commonly used labels in LFA. Colloidal gold is inert and gives very perfect spherical particles. These particles have very high affinity toward biomolecules and can be easily functionalized. Optical properties of gold nanoparticles are dependent on size and shape. Size of particles can be tuned by use of suitable chemical additives. Their unique features include environment friendly preparation, high affinity toward proteins and biomolecules, enhanced stability, exceptionally higher values for charge transfer and good optical signaling. Optical properties of gold nanoparticle enhance sensitivity of analysis in LFA. Sensitivity is a function of molar absorption coefficient and accumulation of gold nanoparticles on target molecule. Optical signal of gold nanoparticles in colorimetric LFA can be amplified by deposition of silver, gold nanoparticles and enzymes.

Use of magnetic particles as colored labels in LFA has been reported by number of researchers. Colored magnetic particles produce color at the test line which is measured by an optical strip reader but magnetic signals coming from magnetic particles can also be used as detection signals and recorded by a magnetic assay reader. It has been reported that magnetic signals are stable for longer time compared to optical signals and they enhance sensitivity of LFA by 10 to 1000 folds

Fluorescent molecules are widely used in LFA as labels and the amount of fluorescence is used to quantitate the concentration of analyte in the sample. Detection of proteins is accomplished by using organic fluorophores such as rhodamine as labels in LFA. High photostability and brightness are required for LFAs.

Quantum dots are also used in LFAs. These semiconducting particles are not only water soluble but can also be easily combined with biomolecules because of closeness in dimensions. Owing to their unique optical properties, quantum dots have come up as a substitute to organic fluorescent dyes. Like gold nanoparticles QDs show size dependent optical properties and a broad spectrum of wavelengths can be monitored. Single light source is sufficient to excite quantum dots of all different sizes. QDs have high photostability and absorption coefficients. They can retain their fluorescent properties within the cells and bodies of organisms and less susceptible to metabolic degradation because of their inorganic nature.

Upconverting phosphors (UCP) are also labels which find use in LFAs. UPA labels are characterized by their excitation in infra-red region and emission in high energy visible region. Compared to other fluorescent materials, they have a unique advantage of not showing any auto fluorescence. Because of their excitation in IR regions, they do not photo degrade biomolecules. A major advantage lies in their production from easily available bulk materials. UCP particles were found to show size dependent sensitivity and specificity for detection of antibodies using LFA in sera of patients.

Enzymes are also employed as labels in LFA. But they increase one step in LFA which is application of suitable substrate after complete assay. This substrate will produce color at test and control lines as a result of enzymatic reaction. Horse-radish peroxidase labeled antibody conjugates can be used for detection of primary animal IgGs. In case of enzymes, selection of suitable enzyme substrate combination is one necessary requirement in order to get a colored product for strip reader or electroactive product for electrochemical detection. In other words, sensitivity of detection is dependent on the enzyme/substrate combination. Enhanced LFA sensitivity was observed when enzyme loaded gold nanoparticles were used as a label.

Colloidal carbon is comparatively inexpensive LFA label and its production can be easily scaled up. Because of their black color, carbon NPs can be easily detected with high sensitivity. Colloidal carbon can be functionalized with a large variety of biomolecules for detection of low and high molecular weight analytes. Carbon black nanoparticles showed very low detection limits compared to other labels. The sensitivity of LFA employing colloidal carbon is reported to be comparable with ELISA assay.

In case of gold nanoparticles or other color producing labels, qualitative or semi-quantitative analysis can be done by visual inspection of colors at test and control lines. The major advantage of visual inspection is rapid qualitative answer in “Yes” or “NO”. Such quick replies about presence of an analyte in clinical analysis have very high importance. Such tests can help doctors or other investigators to make an immediate decision, e.g., situations where test results from central labs cannot be waited for because of huge time consumption. But for quantification, optical strip readers are employed for measurement of the intensity of colors produced at test and control lines of strip. This is achieved by inserting the strips into a strip reader and intensities are recorded simultaneously by imaging software. Optical images of the strips can also be recorded with a camera and then processed by using a suitable software. Such systems use monochromatic light and wavelength of light can be adjusted to get a good contrast among test and control lines and background. Automated systems have advantages over manual imaging and processing in terms of time consumption, interpretation of results and adjustment of variables. In case of fluorescent labels, a fluorescence strip reader is used to record fluorescence intensity of test and control lines. Fluorescence brightness of a test line increases with an analyte's concentration in the sample. Magnetic strip readers and electrochemical detectors are also reported as detection systems in LFTS but they are not as common. Selection of detector is mainly determined by the label employed in analysis.

LFA strips give qualitative or semi-quantitative results which can be observed by naked eyes. Conventional LFAs are normally qualitative and give answers as a ‘yes’ or ‘no’ result. A good LFA biosensor should have the following properties: biocompatibility, high specificity, high sensitivity, rapidity of analysis, reproducibility/precision of results, wide working range of analysis, accuracy of analysis, high through-put, compactness, low cost, simplicity of operation, portability, flexibility in configuration, possibility of miniaturization, potential of mass production and on-site detection.

A sequencing library can be generated from a sample using the methods, compositions and kits provided herein or any suitable methods known in the art. Various commercial kits exist for the preparation of samples for NGS (e.g., Ion Ampliseq Library Kit 2.0, ThermoFisher Scientific, Catalog No.: 4475345). A sequencing library preferably comprises a plurality of target nucleic acids (e.g., a multiplex) that is compatible with any of the sequencing systems disclosed herein or known in the art. In some embodiments, a sequencing library generated from a sample from a subject is prepared for use on an Illumina sequencing platform (e.g., HiSeq or MiSeq). Optionally, target nucleic acids prepared for use in the sequencing library may comprise one or more adapters appended to one, or both, ends of the target nucleic acid molecules to aid in downstream analysis or classification. Optionally, the target nucleic acid molecules of the sequencing library may contain a barcode to distinguish one set of target nucleic acid molecules from a first sample from target nucleic acid molecules prepared from a second (e.g., a different sample from a different source or a sample collected at a different time from the same source (e.g., before and after infection) sample.

Steps for preparing a library preparation may include one or more of: obtaining (e.g., isolating or extracting) target nucleic acids from a sample, fragmenting the target nucleic acids, amplify the target nucleic acid using one or more primers thereby forming a library preparation, and storing the library preparation for later use. The library preparation steps outlined above are applicable to both DNA and RNA based libraries. Typically to amplify RNA, the target RNA is incubated with a DNA destroying reagent (e.g., DNase) to obtain an RNA sample. Steps for preparing a sequencing preparation may include one or more of: amplify the target nucleic acid molecules of the library preparation, attaching adapters to the amplified library preparation, and sequencing the amplified library preparation on a sequencing platform.

Any detection method may be used which is suitable for the sequencing assay employed. In some embodiments, the sequencing assay can employ a label in the detection method. The term “label” as used herein refers to a composition detectable by spectroscopic, photochemical, biochemical, immunochemical, chemical, or other physical means. For example, useful labels include fluorescent dyes, luminescent agents, radioisotopes (e.g., ³²P, ³H), electron-dense reagents, enzymes, biotin, digoxigenin, or haptens and proteins, or other entities which can be made detectable, e.g., by incorporating a radiolabel into an oligonucleotide, peptide, or antibody specifically reactive with a target molecule. Exemplary detection methods include radioactive detection (e.g., ³²P), optical absorbance detection, e.g., UV-visible absorbance detection, optical emission detection, e.g., fluorescence or chemiluminescence. For example, labeled amplification products from a PCR, such as cDNA or DNA, can be detected using a sequencing platform by scanning all or portions of each labeled amplification product simultaneously or serially, depending on the sequencing platform and method used. For radioactive signals (e.g., ³²P), a phosphorimager device can be used (Johnston et al., 1990; Drmanac et al., 1992; 1993). In another embodiment, target molecules (e.g., cDNA molecules) can be label-free and their production detected by release of hydrogen ions during incorporation of each nucleotide during DNA synthesis (i.e., polymerization of DNA) (See, Ion Torrent sequencing platforms such as Personal Genome Machine and Proton sequencers, Life Technologies Corp., Carlsbad, Calif. and e.g., U.S. Pat. Nos. 9,139,874; 9,309,557 and 9,657,281). In another embodiment, the sequencing assay can include nanopore sequencing such as, but not limited to, sequencing methods disclosed in U.S. Pat. Nos. 8,852,864; 8,968,540; 9,121,059; 9,279,153; and 9,542,527.

In some embodiments, a signal from any of the detection methods utilized can be measured and/or analyzed manually or by appropriate computational methods to formulate results. The results can be measured to provide qualitative or quantitative results, depending on the needs of the user. Reaction conditions can include appropriate controls for verifying the integrity of amplification and/or sequencing assay, and for providing standard curves for quantitation, if desired (e.g., RT-qPCR). In some embodiments, a computational method comprises a computer system.

In some embodiments, the sequencing assay comprises a polymerase chain reaction (PCR). In one embodiment the sequencing assay comprises quantitative PCR (qPCR), reverse-transcription polymerase chain reaction (RT-PCR), or reverse transcription quantitative polymerase chain reaction (RT-qPCR).

In some embodiments, data obtained from the sequencing assay is in form of nucleotide sequences representing sequence reads obtained from the sample. In one embodiment, the sequencing assay comprises at least one primer selected from any of SEQ ID NOs: 1-8 for bacterial detection; SEQ ID NOs: 9-15 for Babesia detection; SEQ ID NOs: 16-23 for mycobacterium detection and SEQ ID NOs: 24-29 for fungi detection. It will be readily apparent that where co-infection or multiple organism may be present any combination of SEQ ID NOs: 1-29 may be used. In another embodiment, the sequencing assay comprises at least one forward primer selected from any of the forward primers in Table 1 or at least one reverse primer selected from any of the reverse primers in Table 1. In another embodiment, where the sample is suspected of containing a bacterium, at least one of the primers in the sequencing assay comprises a primer that is bacterium universal primer. In one embodiment, the sequencing assay further comprises a probe to determine the amount of amplified product produced in the sequencing assay by the primers. In one embodiment the amount of amplified product produced in the sequencing assay can be measured, determined or quantified by qPCR.

In some embodiments, the sequencing assay produces between 10,000 and 100 million raw sequencing reads. In some embodiments, the sequencing reads can be refined to remove bad quality or low-quality sequencing reads. In some embodiments, the sequencing assay provides greater than 10 sequencing reads and fewer than 100,000 sequencing reads per amplified target nucleic acid. In another embodiment, the sequencing reads can be deduplicated to remove duplicate reads from the raw sequencing assay data.

Any suitable method, calculation, or threshold may be used to determine whether the alignment of the first portion of the sequencing reads corresponds to the first reference genome. In one embodiment, the particular taxon of pathogenic microorganisms may be determined as present in the sample if at least 1%, 2%, 5%, 10% or more, of a first portion of the sequencing reads aligns with the first reference genome. Conversely, any suitable method, calculation or threshold may be used to determine whether a lack of alignment between the first portion of the sequencing reads and the first reference genome corresponds to a lack of the taxon of pathogenic microorganisms in the sample. For example, it may be determined that the target is absent from the sample, where greater than 95%, 96%, 97%, 98%, 99% or more of the sequencing reads do not align with the first reference genome.

Any suitable method, calculation or threshold may be used to determine whether the alignment of the second portion of the sequencing reads corresponds to the second reference genome. In one embodiment, the different taxon of pathogenic microorganisms may be determined as present in the sample if at least 1%, 2%, 5%, 10% or more, of a second portion of the sequencing reads align with the second reference genome. Conversely, any suitable method, calculation or threshold may be used to determine whether a lack of alignment between the second portion of the sequencing reads and the second reference genome corresponds to the different taxon of pathogenic microorganisms in the sample. For example, it may be determined that the different taxon of pathogenic microorganisms is absent from the sample, where greater than 95%, 96%, 97%, 98%, 99% or more of the sequencing reads do not align with the second reference genome.

The methods, compositions and kits disclosed herein contain primers that are useful for detection of microorganisms in a sample. The primers are suitable for the detection of a plurality of pathogenic microorganisms in a single sample. For example, the primers are “universal” and sufficient to detect all organisms in a particular taxa (e.g., “bacteria” using SEQ ID Nos: 1-8; “babesia” using SEQ ID Nos: 9-15; “mycobacteria” using SEQ ID Nos: 16-23; and “fungi” using SEQ ID Nos:24-29). The primers may be used in a single sequencing assay to determine whether a taxon of pathogenic microorganisms is present in the sample. In another embodiment, the primers or primer pairs may be used in a single sequencing assay to determine whether a plurality of pathogen taxa are present in a single sample. In some instances, each primer (or primer pair) is specific for an individual microbial taxa. In another embodiment, the primers (or primer pairs) may be used to distinguish between taxa within a single taxonomic classification (e.g., bacterial domain or fungal domain).

In some embodiments, the method, kits and compositions disclosed herein comprise one or more additional primers distinct from the primers identified in Table 1. These additional primers can be random primers that are selected without regard to the pathogen of interest to be detected (e.g., random hexamers (N₆) or random nonamers (N₉)). In one embodiment, the additional primers are random primers having a length of less than ten nucleotides. The additional primers can optionally include one or more modified nucleotides/nucleosides or nucleotide analogs. However, typically the additional primers retain conventional hydrogen base-pair bonding capabilities. In some embodiments, the additional primers are designed to hybridize to a target sequence in the sample (e.g., particular taxon of pathogens) and are present in an excess as compared to the primers of Table 1 (e.g., in the amplification reaction). In other embodiments, the primers of Table 1 are present in excess compared to the additional primers. For example, the primers can be present in a 2:1, 3:1, 4:1, 5:1, 6:1, 7:1, 8:1, 9:1, 10:1, or greater ratio as compared to the additional primers (e.g., random primers). In one embodiment, the primers of the disclosure are present in a 5:1 ratio (e.g., forward primer ratio 5:reverse primer ratio 5:random primer ratio 1). In another embodiment, the primers of the disclosure are present in a 10:1 ratio (e.g., forward primer ratio 5:reverse primer ratio 5:random primer ratio 1).

In some embodiments, a sample is screened for a particular taxon of microorganisms by incubating the sample with primers from Table 1 that are optionally ligated or tagged to a nucleic acid adapter under suitable conditions (e.g., hybridization and amplification conditions) such that a plurality of amplified target nucleic acid molecules are generated (e.g., cDNA or DNA molecules). In some embodiments, the primers (see, e.g., SEQ ID NOs: 1-29) are combined with PCR reagents under reaction conditions that induce primer extension. For example, primer extension reactions generally include KCl, Tris-HCl, MgCl₂, denatured template nucleic acid, primer, and a polymerase or reverse transcriptase. The PCR usually contains dNTPs, such as dATP, dCTP, dTTP, dGTP, or one or more analogs thereof.

In some embodiments, the method further comprises incubating the sample in the presence of one or more random primers that are optionally ligated or tagged with the same (or different) nucleic acid adapter. In one embodiment, the method comprises generating a complementary DNA (cDNA) sequence to a target nucleic acid molecule (which corresponds to a particular taxon of pathogens) by reverse transcribing the target nucleic acid molecule by hybridizing one or more of the primers to a complementary nucleic acid sequence present in the sample. In one embodiment, the method further comprises amplifying the cDNA molecules using a nucleic acid adapter in a subsequent amplification reaction. In another embodiment, the cDNA molecules can be directly sequenced using any sequencing assay known in the art to obtain sequencing reads.

In one embodiment, a sample is screened for a particular taxon of pathogenic microorganisms by incubating the sample with a set of primers of Table 1, optionally ligated to a nucleic acid adapter, optionally in the presence of one or more random primers, optionally ligated to the same nucleic acid adapter, thereby allowing the primers to hybridize to a complementary nucleic acid sequence in the sample; extending the primers in a template dependent manner thereby generating cDNA; and optionally amplifying the cDNA to obtain a sequencing library. In some embodiments, the sequencing library can be sequenced using any method available in the art to obtain sequencing reads. In one embodiment, the sequencing reads can be filtered to remove adapter nucleic acid sequences, low-quality and/or low-complexity sequences.

In some embodiments, the methods (and associated kits and compositions) comprise one or more probes. The term “probe” as used herein refers to a molecule (e.g., a protein, nucleic acid, aptamer, etc.,) that interacts with or binds to a target. Non-limiting examples of molecules that specifically interact with or specifically bind to a target include nucleic acids (e.g., oligonucleotides or magnetic beads coated with oligonucleotides), proteins (e.g., antibodies, transcription factors, zinc finger proteins, non-antibody protein scaffolds, etc.,) and aptamers. Binding typically indicates that the probe binds a majority of the target, assuming an appropriate molar ratio of probe to target. For example, a probe that binds a target molecule typically binds to at least 2/3 of the target molecules in a solution (e.g., 67%, 75%, 80%, 85%, 90%, 91%, 92%, 93%, 94%, 95%, 96%, 97%, 98%, 99%, or 100%). In another embodiment, a probe binds to a target molecule with at least 2-fold greater affinity than non-target molecules, e.g., at least 4-fold, 5-fold, 6-fold, 7-fold, 8-fold, 9-fold, 10-fold, 20-fold, 25-fold, 50-fold, or 100-fold greater affinity. One of skill will recognize that some variability will arise depending on the method and/or threshold of determining binding.

In some embodiments, the probe can comprise one or more moieties that allow for fluorescent detection of the probe when bound to or interacting with the target. In some embodiments, one or more probes can be added to the sequencing assay optionally, after formation of cDNA molecules (e.g., library preparation) to “pull down” targets having a complementary nucleic acid sequence. In one embodiment, the probe is a bait capture probe (see, e.g., Penalba et al., Mol. Ecol. Res., (2014) 14:1000-10; and xGen target capture probes commercially available from Integrated DNA Technologies, Iowa). In some embodiments, the probes allow for selective enrichment of the target molecules from the sample. In some embodiments, the probe can be attached to a magnetic bead and/or biotinylated.

The disclosure also contemplates compositions which are useful in practicing the disclosure. Such compositions may include one or more primers or probes disclosed herein. Optionally, the compositions may further include an adapter.

In one embodiment, the disclosure generally relates to a nucleic acid molecule for detecting a target sequence from a particular taxon of pathogenic microorganisms comprising a primer that is complementary or substantially complementary to the target sequence, wherein the primer as set forth in Table 1. In some embodiments, the composition further comprises an adapter located 5′ of the primer.

In some embodiments, a composition comprising a reaction mixture containing at least one of the primers set forth in Table 1 and a target sequence is also contemplated.

The disclosure also contemplates kits which are useful in practicing the disclosure. Such kits may include one or more primers or probes as disclosed herein. Optionally, the kits may include additional primers, probes, instructions, or vessels for one or more components of the kit. The kit may also include buffers and any other reagents that facilitate the method.

In one embodiment, the disclosure provides a kit for detecting the presence of a pathogen in a sample based on the presence of a sequencing read derived from the sample. In some embodiments, a first portion of the sequencing read aligns with a first reference genome, which corresponds to a particular taxon of pathogens. In some embodiments, a second portion of the sequencing read aligns with a second reference genome, which corresponds to a different taxon of pathogens.

In one embodiment, the disclosure generally relates to a kit comprising at least one primer set forth in Table 1. In one embodiment, the kit is based on the presence or absence of a target sequence (or complement thereof) corresponding to a nucleic acid sequence present in the genome of a particular taxon of pathogens. In one embodiment, the target sequence corresponds to a reverse transcriptase (RT) region of a gene present in the genome of a particular taxon of pathogens.

In some embodiments, presence of the taxon of pathogenic microorganisms is determined by amplifying a region of a gene from the particular taxon of pathogenic microorganisms using universal primers, and aligning a first portion of the target sequence against a first reference genome, wherein the universal primers are any of the primers set forth in Table 1.

In one embodiment, the kit further comprises an adapter. In one embodiment, the adapter is positioned 5′ of the primer. In one embodiment, the kit further comprises one or more additional primers and/or probes. In one embodiment, the additional primers can comprise a random hexamer or a random nonamer. In one embodiment, the one or more probes can be included.

In some embodiments, each of the primers is provided in a separate container, and the kit further includes an additional container having additional primers that are non-specific to the particular taxon of pathogenic microorganisms or different taxon of pathogenic microorganisms or random primers. In another embodiment, a solution or dry mix of pooled primers is provided in a single container, and the kit further includes additional primers (e.g., in the same or different container) that are non-specific to the particular taxon of pathogenic microorganisms or different taxon of pathogenic microorganisms or random primers.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

All patents, patent applications, and publications mentioned herein are incorporated herein by reference in their entireties for all purposes.

1. An isolated oligonucleotide selected from the group consisting of: (i) a sequence of any one of SEQ ID NO:1-29, having 1-5 nucleotides added or removed from the 5′ and/or 3′ ends; and (ii) a sequence consisting of any one of SEQ ID NO:1-29.

2. A composition for microbial detection, the composition comprising at least one oligonucleotide having the sequence set forth in any one of SEQ ID NOs: 1-29.

3. The composition of claim 2, wherein the at least one oligonucleotide comprises at least two or more oligonucleotides.

4. The composition of claim 2, wherein the at least one oligonucleotide is selected from oligonucleotides having the sequence of SEQ ID NOs:1-7, 8, or any two or more of SEQ ID NOs:1-8.

5. The composition of claim 2, wherein the composition is used to detect bacteria.

6. The composition of claim 2, wherein the at least one oligonucleotide is selected from oligonucleotides having the sequence of SEQ ID NOs:9-14, 15, or any two or more of SEQ ID NOs:9-15.

7. The composition of claim 2, wherein the composition is used to detect babesia.

8. The composition of claim 2, wherein the at least one oligonucleotide is selected from oligonucleotides having the sequence of SEQ ID NOs:16-22, 23, or any two or more of SEQ ID NOs:16-23.

9. The composition of claim 2, wherein the composition is used to detect mycobacteria.

10. The composition of claim 2, wherein the at least one oligonucleotide is selected from oligonucleotides having the sequence of SEQ ID NOs:24-28, 29, or any two or more of SEQ ID NOs:24-29.

11. The composition of claim 2, wherein the composition is used to detect fungi.

12. A composition for detecting a microbe selected from the group consisting of bacteria, mycobacteria, babesia, fungi and any combination thereof, the composition comprising at least one primer having a sequence selected from the group consisting of SEQ ID NO:1-29 and any combination thereof.

13. A method of claim 12, wherein the method is for detecting the presence of a bacterial species in a sample, the method comprising contacting the sample with at least one universal primer having a sequence set forth in any one of SEQ ID NOs: 1-8.

14. A method of claim 12, wherein the method is for detecting the presence of a babesia species in a sample, the method comprising contacting the sample with at least one universal primer having a sequence set forth in any one of SEQ ID NOs: 9-15.

15. A method of claim 12, wherein the method is for detecting the presence of a mycobacterial species in a sample, the method comprising contacting the sample with at least one universal primer having a sequence set forth in any one of SEQ ID NOs: 16-23.

16. A method of claim 12, wherein the method is for detecting the presence of a fungal species in a sample, the method comprising contacting the sample with at least one universal primer having a sequence set forth in any one of SEQ ID NOs: 24-29.

17. A method for determining microbial content in a sample, said method comprising amplifying a target nucleotide sequence which is substantially conserved amongst two or more species of microorganisms, said amplification being for a time and under conditions sufficient to generate a level of an amplification product such that the presence of the microbe can be detected, wherein the method uses at least one primer selected from SEQ ID NOs:1-29.

18. The method according to claim 17, wherein said target nucleotide sequence is selected from the group consisting of DNA, RNA, ribosomal DNA (rDNA) and ribosomal RNA (rRNA).

19-21. (canceled)

22. The method according to claim 18, wherein the rDNA or rRNA is 16S rDNA or rRNA.

23. (canceled)

24. The method according to claim 18, wherein the sample is a biological, medical, agricultural, industrial or environmental sample.

25. (canceled)

26. The method according to claim 17, wherein the amplification uses a primer having the sequence selected from SEQ ID NO:1-8 or a sequence having from 1-5 additional nucleotides at the 5′ and/or 3′ end of any of the sequence of SEQ ID NO:1-8 and wherein the microbial content is bacteria.

27. The method according to claim 17, wherein the amplification uses a primer having the sequence selected from SEQ ID NO:9-15 or a sequence having from 1-5 additional nucleotides at the 5′ and/or 3′ end of any of the sequence of SEQ ID NO:9-15 and wherein the microbial content is babesia.

28. The method according to claim 17, wherein the amplification uses a primer having the sequence selected from SEQ ID NO:16-23 or a sequence having from 1-5 additional nucleotides at the 5′ and/or 3′ end of any of the sequence of SEQ ID NO:16-23 and wherein the microbial content is mycobacteria.

29. The method according to claim 17, wherein the amplification uses a primer having the sequence selected from SEQ ID NO:24-29 or a sequence having from 1-5 additional nucleotides at the 5′ and/or 3′ end of any of the sequence of SEQ ID NO:24-29 and wherein the microbial content is fungi.

30. A kit in compartmental form, said kit comprising a compartment adapted to contain one or more primers having a sequence selected from SEQ ID NOs:1-29, and any combination thereof, capable of participating in an amplification reaction of DNA comprising or associated with 16S rDNA or 16S rRNA, and optionally another compartment adapted to contain reagents to conduct an amplification reaction.
